SEQUENCE LISTING (2 sequences)

SEQ ID NO: 1 - protein, 67 aa, Ixodes ricinus
ANHKGRGRPAKCKLPPDDGPCRARIPSYYFDRKTKTCKEFMYGGCEGNENNFENITTCQEECRAKKV

SEQ ID NO: 2 - protein, 67 aa, synthetic construct
ANHKGRGRPAKCKLPPDDGPCRARIPSYYFDRKTKTCKEFMYGGCEGNENNFEQITTCQEECRAKKV

(19) United States
(12) Patent Application Publication
(10) Pub. No.: US 2026/0364144 A1
(43) Pub. Date: Nov. 19, 2026

(54) STABLE IR-CPI COMPOSITIONS WITH EXTENDED SHELF-LIFE

(71) Applicant: BIOXODES, Charleroi (BE)

(72) Inventors: Stéphanie DEMOULIN, Charleroi (BE); Sandrine DEROCHETTE, Charleroi (BE); Marc DECHAMPS, Charleroi (BE)

(73) Assignee: BIOXODES, Charleroi (BE)

(21) Appl. No.: 19/194,323

(22) Filed: Apr. 30, 2025

(51) Int. Cl. A61K 38/17; A61K 47/02; A61K 47/18; C07K 14/435

(52) U.S. Cl. CPC A61K 38/1767; A61K 47/02; A61K 47/183; C07K 14/43527

(57) ABSTRACT

Pharmaceutical compositions including an Ir—CPI (also referred to as BIOX-101) polypeptide as an active ingredient and a stabilizing agent to prevent the aggregation and/or opalescence of the Ir—CPI polypeptide, thereby increasing shelf-life of the active agent. Also, an injectable solution including such pharmaceutical composition and a physiological liquid or a saline solution.

### REFERENCE TO A SEQUENCE LISTING

The entire contents of the electronic sequence listing (“2086 PCT-Sequence listing.xml”; Size: 2,769 bytes; and Date of Creation: Apr. 28, 2025) is herein incorporated by reference in its entirety.

### FIELD

The present invention relates to pharmaceutical compositions comprising stabilizing agents, in particular to increase the stability of a polypeptide.

### BACKGROUND

Thrombotic disorders, such as stroke, remain a leading cause of morbidity and mortality worldwide. These conditions are characterized by the formation of blood clots that obstruct normal blood flow, leading to tissue damage and organ dysfunction. Thrombotic events, especially in the brain, are frequently accompanied by an inflammatory response, a phenomenon often described as thromboinflammation. This term refers to the intricate and bidirectional interaction between the coagulation system and the innate immune response, notably the activation of neutrophils and the formation of neutrophils extracellular traps (NETosis). Indeed, following vascular injury or ischemia, immune cells such as neutrophils are recruited to the site of injury, releasing pro-inflammatory cytokines, neutrophil extracellular traps and tissue factor, which in turn promote further thrombin generation and platelet activation. This creates a feed-forward loop that exacerbates tissue damage and complicates recovery.

The Applicant previously demonstrated that polypeptides isolated from the salivary gland of the tick I. ricinus, Ir—CPI (Ixodes ricinus-Contact Phase Inhibitor), specifically target coagulation Factors XIa and XIIa (WO2009141382; Decrem et al., 2009). Ir—CPI was thus found to be a specific inhibitor of the intrinsic coagulation pathway with antithrombotic effect, and enables the treatment of thrombotic conditions.

The Applicant has further found that Ir—CPI possesses anti-inflammatory properties, and unexpectedly inhibits the recruitment of polymorphonuclear neutrophils (PMNs) and platelets, and also inhibits the activation of PMNs and the formation of neutrophil extracellular traps, also called NETosis (WO2019149906). Interestingly, Ir—CPI has no effect on the kallikrein-kinin pathway, indicating that the effects of Ir—CPI on inflammation are mediated by neutrophils activation and NETosis, but not by kallikrein or bradykinin. Ir—CPI is thus useful to treat thromboinflammation.

The Applicant has further found that Ir—CPI can be used to treat brain inflammation following a brain injury (WO2023222871). Indeed, neuroinflammation or brain inflammation may occur after brain injury, for example in case of hemorrhagic stroke: brain hemorrhage not only causes primary brain injury through direct mechanical effects of the hemorrhage but also leads to secondary brain injury (SBI) including brain inflammation, which is a major contributor to poor outcomes after brain hemorrhage. The Applicant surprisingly demonstrated that Ir—CPI can be safely used to treat brain inflammation following a brain hemorrhage, without causing bleeding or worsening the hemorrhage.

Therefore, Ixodes ricinus salivary gland polypeptides are useful for the treatment and/or the prevention of diseases and conditions associated with the activation of the intrinsic coagulation pathway, such as, for example, thrombosis, for the treatment and/or the prevention of inflammatory diseases with thrombotic tendency and thromboinflammation, and for the treatment of brain inflammation following a brain injury.

The applicant has however observed that this polypeptide in PBS has a limited stability, opalescence appearing after a certain time of storage at temperatures above freezing point. In the context of, e.g., stroke, therapeutic windows are narrow and side effect profiles must be tightly controlled, it is therefore useful to have a formulation of the active principle that is stable and readily available.

There is thus a need to increase stability and shelf-life of Ir—CPI.

The present invention thus relates to a new formulation of antithrombotic proteins comprising an Ixodes ricinus salivary gland polypeptide having a longer shelf-life, and their use for treating and/or preventing diseases and conditions associated with the activation of neutrophils and/or NETosis.

### SUMMARY

The present invention relates to a pharmaceutical composition comprising:
a protein or polypeptide comprising a polypeptide that has at least 75% sequence identity with the amino acid sequence SEQ ID NO: 2, preferably a protein or polypeptide having the amino acid sequence of SEQ ID NO: 2, and a stabilizing agent of formula (I) to prevent aggregation and/or opalescence

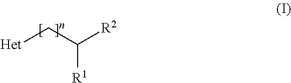

wherein R¹ is H, NH₂ or alanine; R² is H or COOH;
n=0, 1 or 2;
Het is a nitrogen containing heterocycle selected from phenyl, 4-hydroxyphenyl, pyrazol-3-yl, pyrazol-4-yl, pyrazol-5-yl, imidazole-4-yl, 1,2,4-Triazol-3-yl, indol-3-yl, 4-pyridyl, 3-pyridyl, 2-pyridyl.

In a preferred embodiment, the stabilizing agent is histidine, preferably L-histidine, i.e. Het is imidazole-4-yl, n=1 and R¹═NH₂ and R²═COOH.

The pharmaceutical composition of the invention can be diluted for intravenous administration to patients. The invention therefore also relates to an injectable solution comprising the pharmaceutical composition as defined above diluted with a physiological liquid or saline solution.

In one embodiment, the injectable solution comprises
a polypeptide that has at least 75% sequence identity with the amino acid sequence SEQ ID NO: 2, and
a stabilizing agent of formula (I) to prevent aggregation and/or opalescence

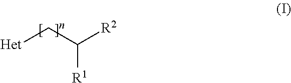

wherein R¹, R², n and Het are as described above, and
a physiological liquid or saline solution.

In some embodiments, the stabilizing agent is an amino acid, preferably an amino acid in L-configuration.

In some embodiments, the stabilizing agent is histidine, preferably L-histidine.

In some embodiments, the concentration of the stabilizing agent is comprised between 1 mM and 50 mM, preferably between 10 mM and 40 mM, preferably between 15 mM and 30 mM.

In some embodiments, the pharmaceutical composition has a pH of 5.0 to 7.5, preferably of 5.5 to 7.0.

In some embodiments, the protein or polypeptide comprising a polypeptide that has at least 75% sequence identity with the amino acid sequence SEQ ID NO: 2 is present at a concentration comprised between 15 mg/mL and 35 mg/mL, preferably between 20 mg/mL and 30 mg/mL, preferably around 25 mg/mL.

In some embodiments, in the pharmaceutical composition of the invention, the protein or polypeptide has the amino acid sequence SEQ ID NO: 2; and the stabilizing agent is histidine.

In another embodiment, the pharmaceutical composition of the invention is provided as a kit for reconstitution of an injectable solution. The invention therefore also relates to a kit for reconstitution of an injectable solution comprising:
the pharmaceutical composition as defined above and
a physiological liquid or saline solution.

The invention also relates to the method for reconstituting the injectable solution from the kit, comprising mixing the pharmaceutical composition with the physiological liquid.

The invention also relates to the use of a stabilizing agent as defined above for stabilizing a protein or polypeptide comprising a polypeptide that has at least 75% sequence identity with the amino acid sequence SEQ ID NO: 2.

In a preferred embodiment, the invention relates to the use of histidine, and in particular L-histidine for stabilizing a protein or polypeptide comprising a polypeptide that has at least 75% sequence identity with the amino acid sequence SEQ ID NO: 2 and preferably for stabilizing the polypeptide that has the amino acid sequence SEQ ID NO: 2.

The invention further relates to the pharmaceutical composition, the injectable solution or the kit of the invention, for use as a medicament.

The invention further relates to a medicament comprising the pharmaceutical composition of the invention.

The invention further relates to the medical use of the pharmaceutical composition, the injectable solution or the kit as defined above.

The invention also relates to a method of treatment comprising administering to a patient the injectable solution as defined above.

Ir—CPI is useful for inhibiting the activity and/or content of Factor XIIa and Factor XIa and for inhibiting platelet recruitment, neutrophil recruitment, neutrophil activation and/or neutrophil extracellular trap formation (NETosis).

The present invention further relates to the pharmaceutical composition, the injectable solution, or the kit as described herein, for use for preventing and/or treating a disease, preferably a disease selected from the group consisting of:
thrombotic disorders, preferably selected from the group consisting of venous thrombosis such as deep vein thrombosis, pulmonary embolism, renal vein thrombosis, and hepatic vein thrombosis; arterial thrombosis such as myocardial infarction and peripheral artery disease; and thromboembolism;
cerebrovascular diseases, preferably selected from the group consisting of ischemic stroke, hemorrhagic stroke such as intracerebral hemorrhage (ICH) and subarachnoid hemorrhage (SAH), thrombus formation following cerebral injuries, and cerebral ischemia;
atherosclerosis;
conditions associated with thrombosis such as preeclampsia, eclampsia, and HELLP syndrome;
inflammatory disorders associated with thrombosis such as rheumatoid arthritis, sepsis, and inflammatory bowel disease;
thromboinflammation;
brain inflammation, preferably brain inflammation occurring after a brain injury and/or a brain hemorrhage such as intracerebral hemorrhage (ICH), more preferably wherein the pharmaceutical composition, the injectable solution, or the kit as described herein is administered within 120 hours, 96 hours or 72 hours after the onset of brain injury and/or brain hemorrhage; and
neuronal cell death, preferably neuronal cell death occurring after a brain injury and/or a brain hemorrhage such as intracerebral hemorrhage (ICH), more preferably wherein the pharmaceutical composition, the injectable solution, or the kit as described herein is administered within 120 hours, 96 hours or 72 hours after the onset of brain injury and/or brain hemorrhage.

Thus, the present invention further relates to the pharmaceutical composition, injectable solution, or the kit as described herein, for use for preventing and/or treating a disease, preferably a disease selected from the group consisting of:
a thrombotic disorder,
a cerebrovascular disease,
inflammatory disorders associated with thrombosis
thromboinflammation,
conditions associated with thrombosis,
a disease associated with platelet recruitment, neutrophil recruitment, neutrophil activation and/or NETosis,
brain inflammation, and
neuronal cell death.

The present invention further relates to the pharmaceutical composition, injectable solution, or the kit as described herein, for use for preventing and/or treating a disease, preferably a disease selected from the group consisting of:
thrombotic disorders, preferably selected from the group consisting of venous thrombosis such as deep vein thrombosis, pulmonary embolism, renal vein thrombosis, and hepatic vein thrombosis; arterial thrombosis such as myocardial infarction and peripheral artery disease; and thromboembolism;
cerebrovascular diseases, preferably selected from the group consisting of ischemic stroke, hemorrhagic stroke such as intracerebral hemorrhage (ICH) and subarachnoid hemorrhage (SAH), thrombus formation following cerebral injuries, and cerebral ischemia;
atherosclerosis;
conditions associated with thrombosis such as preeclampsia, eclampsia, and HELLP syndrome; and
inflammatory disorders associated with thrombosis such as rheumatoid arthritis, sepsis, and inflammatory bowel disease;
   preferably the disease is stroke.

In some embodiments, the disease is selected from the group consisting of stroke, thromboembolism, myocardial infarction, cerebrovascular diseases, cerebral ischemia, pulmonary embolism, and thrombosis such as renal vein thrombosis and hepatic vein thrombosis.

In some embodiments, the disease is stroke. In one embodiment, the disease is ischemic stroke. In another embodiment, the disease is hemorrhagic stroke.

In some embodiments, the disease is a disease or condition associated with platelet recruitment, neutrophil recruitment, neutrophil activation and/or NETosis, preferably the disease or condition associated with platelet recruitment, neutrophil recruitment, neutrophil activation and/or NETosis is thromboinflammation.

In some embodiments, the disease is thromboinflammation.

In some embodiments, the disease is thromboinflammation selected from the group consisting of atherosclerosis-induced thromboinflammation, plaque rupture-induced thromboinflammation, devices-induced thromboinflammation, catheterism-induced and/or stent-induced thromboinflammation, extracorporeal circulation-induced thromboinflammation, cerebral injuries-induced thromboinflammation, coronary artery disease-induced thromboinflammation, myocardial infraction-induced acute thromboinflammation, cancer-induced thromboinflammation, metastasis-induced thromboinflammation, stroke-induced thromboinflammation, Behçet's disease (BD)-induced thromboinflammation, antineutrophil cytoplasmic antibody-associated (ANCA) vasculitides-induced thromboinflammation, Takayasu arteritis-induced thromboinflammation, rheumatoid arthritis-induced thromboinflammation, systemic lupus erythematosus-induced thromboinflammation, antiphospholipid syndrome-induced thromboinflammation, familial Mediterranean fever-induced thromboinflammation, thromboangiitis obliterans (TAO)-induced thromboinflammation, sepsis-induced thromboinflammation, inflammatory bowel diseases-induced thromboinflammation, heparin-induced thrombocytopenia-induced thromboinflammation, immunothrombosis-induced thromboinflammation, (pre) eclampsia-induced thromboinflammation, HELLP syndrome-induced thromboinflammation, thromboinflammation complication in cell and cell cluster transplantation and in whole organ transplantation or grafts, venous thromboembolism-induced thromboinflammation, aneurysms-induced thromboinflammation, and skeletal muscle ischemia-reperfusion syndrome-induced thromboinflammation.

In some embodiments, the disease is thromboinflammation selected from the group consisting of cerebral injuries-induced thromboinflammation, cancer-induced thromboinflammation, metastasis-induced thromboinflammation, and stroke-induced thromboinflammation. In some embodiments, the disease is thromboinflammation selected from the group consisting of cerebral injuries-induced thromboinflammation, and stroke-induced thromboinflammation. In some embodiments, the disease is cerebral injuries-induced thromboinflammation. In some embodiments, the disease is stroke-induced thromboinflammation.

In some embodiments, the disease is brain inflammation. In some embodiments, the disease is brain inflammation occurring or susceptible to occur after brain hemorrhage. In some embodiments, the disease is brain inflammation occurring or susceptible to occur after brain injury.

In some embodiments, the pharmaceutical composition, injectable solution, or the kit as described herein, is administered within about 120 hours after the onset of brain inflammation, preferably within 96 hours, more preferably within 72 hours. In some embodiments, the pharmaceutical composition, injectable solution, or the kit as described herein, is administered within about 120 hours after the onset of brain hemorrhage, preferably within 96 hours, more preferably within 72 hours. In some embodiments, the pharmaceutical composition, injectable solution, or the kit as described herein, is administered within about 120 hours after the onset of brain injury, preferably within 96 hours, more preferably within 72 hours.

In some embodiments, the disease is neuronal cell death. In some embodiments, the disease is neuronal cell death occurring or susceptible to occur after brain hemorrhage. In some embodiments, the disease is neuronal cell death occurring or susceptible to occur after brain injury.

In some embodiments, the pharmaceutical composition, injectable solution, or the kit as described herein, is administered within about 120 hours after the onset of brain inflammation, preferably within 96 hours, more preferably within 72 hours. In some embodiments, the pharmaceutical composition, injectable solution, or the kit as described herein, is administered within about 120 hours after the onset of brain hemorrhage, preferably within 96 hours, more preferably within 72 hours. In some embodiments, the pharmaceutical composition, injectable solution, or the kit as described herein, is administered within about 120 hours after the onset of brain injury, preferably within 96 hours, more preferably within 72 hours.

In some embodiments, the pharmaceutical composition, injectable solution, or the kit as described herein, is for use for inhibiting platelet recruitment, neutrophil recruitment, neutrophil activation and/or neutrophil extracellular trap formation (NETosis).

### BRIEF DESCRIPTION OF THE DRAWINGS

FIG. 1 is a graph showing the trend plot of z-average vs Temperature z-average for Formulation 1 (batch B3015310)-temperature ramp from 25 to 70 °C (inset: of z-average vs Temperature z-average 64 to 70 °C). DLS method.

FIG. 2 is a photograph showing the appearance of Ir—CPI (batch B3015310) before and after temperature ramp study showing increased opalescence.

FIG. 3 is a graph showing the PSD by Intensity measured on 8 formulations during thermal stress (50 °C to 80 °C).

### DEFINITIONS

In the present invention, the following terms have the following meanings:

The terms “about” or “around” preceding a value means plus or minus 10% of said value.

The term “amino acid substitution” refers to the replacement in a polypeptide of one amino acid with another amino acid. In one embodiment, an amino acid is replaced with another amino acid having similar structural and/or chemical properties, e.g., conservative amino acid replacements. “Conservative amino acid substitution” may be made on the basis of similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; polar neutral amino acids include glycine, serine, threonine, cysteine, tyrosine, asparagine, and glutamine; positively charged (basic) amino acids include arginine, lysine, and histidine; negatively charged (acidic) amino acids include aspartic acid and glutamic acid. Non-conservative substitutions will entail exchanging a member of one of these classes for another class. For example, amino acid substitutions can also result in replacing one amino acid with another amino acid having different structural and/or chemical properties, for example, replacing an amino acid from one group (e.g., polar) with another amino acid from a different group (e.g., basic). Amino acid substitutions can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful.

The term “at least 75% sequence identity” encompasses 75% sequence identity, 76% sequence identity, 77% sequence identity, 78% sequence identity, 79% sequence identity, 80% sequence identity, 81% sequence identity, 82% sequence identity, 83% sequence identity, 84% sequence identity, 85% sequence identity, 86% sequence identity, 87% sequence identity, 88% sequence identity, 89% sequence identity, 90% sequence identity, 91% sequence identity, 92% sequence identity, 93% sequence identity, 94% sequence identity, 95% sequence identity, 96% sequence identity, 97% sequence identity, 98% sequence identity, 99% sequence identity, and 100% sequence identity.

The term “identity” refers to a measure of the identity of nucleotide sequences or amino acid sequences. In general, the sequences are aligned so that the highest order match is obtained. “Identity” per se has an art-recognized meaning and can be calculated using published techniques. See, e.g.: Computational Molecular Biology, Lesk, A. M., ed., Oxford University Press, New York, 1988; Biocomputing: Informatics And Genome Projects, Smith, D. W., ed., Academic Press, New York, 1993; Computer Analysis Of Sequence Data, Part I, Griffin, A. M., and Griffin, H. G., eds, Humana Press, New Jersey, 1994; Sequence Analysis In Molecular Biology, von Heijne, G., Academic Press, 1987; and Sequence Analysis Primer, Gribskov, M. and Devereux, J., eds, M Stockton Press, New York, 1991. While there exist a number of methods to measure identity between two polynucleotide or polypeptide sequences, the term “identity” is well known to skilled artisans (Carillo and Lipton, SIAM J Applied Math, 1998, 48:1073). Methods commonly employed to determine identity or similarity between two sequences include, but are not limited to, those disclosed in Guide to Huge Computers, Martin J. Bishop, ed., Academic Press, San Diego, 1994; and Carillo and Lipton, SIAM J Applied Math, 1998, 48:1073. Methods to determine identity and similarity are codified in computer programs. Preferred computer program methods to determine identity and similarity between two sequences include, but are not limited to, GCG program package (Devereux et al., J Molec Biol, 1990, 215:403). Most preferably, the program used to determine identity levels was the GAP program, as was used in the Examples below.

As an illustration, by a polynucleotide having a nucleotide sequence having at least, for example, 95% “identity” to a reference nucleotide sequence is intended that the nucleotide sequence of the polynucleotide is identical to the reference sequence except that the polynucleotide sequence may include an average up to five point-mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

The term “polynucleotide” refers to any polyribonucleotide or polydeoxyribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically, double-stranded or a mixture of single- and double-stranded regions. In addition, “Polynucleotide” refers to triple-stranded regions comprising RNA or DNA or both RNA and DNA. The term Polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “Polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

The term “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

The term “protein” refers to a sequence of more than 100 amino acids and/or to a multimeric entity. The proteins of the invention are not limited to a specific length of the product. The term “polypeptide” or “protein” does not refer to or exclude post-expression modifications of the protein, for example, glycosylation, acetylation, phosphorylation and the like, as well as other modifications known in the art, both naturally occurring and non-naturally occurring. Such modifications are well described in basic texts and in more detailed monographs, as well as in a voluminous research literature. Modifications can occur anywhere in a polypeptide or protein, including the peptide backbone, the amino acid side-chains and the amino or carboxyl termini. It will be appreciated that the same type of modification may be present in the same or varying degrees at several sites in a given polypeptide or protein. Also, a given polypeptide or protein may contain many types of modifications. Polypeptides or proteins may be branched as a result of ubiquitination, and they may be cyclic, with or without branching. Cyclic, branched and branched cyclic polypeptides or proteins may result from posttranslational natural processes or may be made by synthetic methods. Modifications include acetylation, acylation, ADP-ribosylation, amidation, covalent attachment of flavin, covalent attachment of a hem moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-linkings, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino of amino acids to proteins such as arginylation, and ubiquitination. See, for instance, “Proteins-structure and molecular properties”, 2nd Ed., T. E. Creighton, W. H. Freeman and Comany, New York, 1993; Wolt, F., “Posttranslational Protein Modifications: Perspectives and Prospects”, Posttranslational covalent modification of proteins, B. C. Johnson, Ed., Academic Press, New York, 1983, pgs. 1-12; Seifter et al., “Analysis for protein modifications and nonprotein cofactors”, Meth Enzymol, 1990, 182:626-646; Rattan et al, “Protein Synthesis: Posttranslational Modifications and Aging”, Ann NY Acad Sci, 1992, 663:48-62. A protein may be an entire protein, or a subsequence thereof.

An “isolated protein or polypeptide” is one that has been identified and separated and/or recovered from a component of its natural environment. In a preferred embodiment, the isolated protein or polypeptide will be purified
(1) to greater than 80, 85, 90, 95% by weight of protein or polypeptide as determined by the Lowry method, and most preferably more than 96, 97, 98, or 99% by weight,
(2) to a degree sufficient to obtain at least 15 residues of N-terminal or internal amino acid sequence by use of a spinning cup sequenator, or
(3) to homogeneity by SDS-PAGE under reducing or non-reducing conditions using Coomassie blue or, preferably, silver staining.

Isolated protein or polypeptide includes the protein in situ within recombinant cells since at least one component of the protein's or polypeptide's natural environment will not be present. Ordinarily, however, isolated protein or polypeptide will be prepared by at least one purification step.

The term “fusion protein” refers to a molecule comprising two or more proteins or fragments thereof linked by a covalent bond via their individual peptide backbones, most preferably generated through genetic expression of a polynucleotide molecule encoding those proteins. The polypeptides forming the fusion protein are typically linked C-terminus to N-terminus, although they can also be linked C-terminus to C-terminus, N-terminus to N-terminus, or N-terminus to C-terminus. The polypeptides of the fusion proteins may be fused in any order.

The term “fused” refers to components that are linked by peptide bonds, either directly or through one or more peptide linkers.

“Ixodes ricinus Contact Phase Inhibitor” also referred to as “Ir—CPI” or “BIOX-101”, was previously described by the inventors in WO2009141382, WO2019149906, and WO2023222871, as well as its uses. The term “native Ir—CPI” or “native BIOX-101” refers to naturally occurring Ir—CPI, as opposed to a “modified Ir—CPI” or “optimized Ir—CPI” or “optimized BIOX-101”, which has been modified from a naturally occurring Ir—CPI, e.g., to alter one or more of its properties such as stability. A modified or optimized Ir—CPI or BIOX-101 polypeptide may for example comprise modifications in the amino acid sequence, e.g., amino acid substitutions, deletions or insertions.

The term “subject” refers to a mammal, preferably a human. In one embodiment, the subject is a man. In another embodiment, the subject is a woman. In one embodiment, a subject may be a “patient”, i.e., a warm-blooded animal, more preferably a human, who/which is awaiting the receipt of, or is receiving medical care or was/is/will be the object of a medical procedure, or is monitored for the development of the disease or condition. In one embodiment, the subject is an adult (for example a subject above the age of 18). In another embodiment, the subject is a child (for example a subject below the age of 18).

The term “therapeutically effective amount” means the level or amount of agent that is aimed at, without causing significant negative or adverse side effects to the target, (1) delaying or preventing disease or condition; (2) slowing down or stopping the progression, aggravation, or deterioration of one or more symptoms of disease or condition; (3) bringing about ameliorations of the symptoms of disease or condition; (4) reducing the severity or incidence of disease or condition; or (5) preventing disease or condition. In one embodiment, a therapeutically effective amount is administered prior to the onset of disease or condition, for a prophylactic or preventive action, and/or in order to reduce the likelihood of occurrence of saif disease. Non-limitative examples of said diseases include thrombotic disorders, cerebrovascular diseases, atherosclerosis, conditions associated with thrombosis, inflammatory disorders associated with thrombosis, thromboinflammation, brain inflammation, and neuronal cell death.

The term “treating” or “treatment” or “alleviation” refers to both therapeutic treatment and prophylactic or preventative measures; wherein the object is to prevent or slow down (lessen) the disease or condition. Those in need of treatment include those already affected with the disease or condition as well as those prone to have the disease or condition or those in whom the disease or condition is to be prevented. A subject or mammal is successfully “treated” for an infection if, after receiving a therapeutic amount of a protein or polypeptide according to the methods of the present invention, the patient shows observable and/or measurable reduction in or absence of one or more of the following: reduction in the number of pathogenic cells; reduction in the percent of total cells that are pathogenic; and/or relief to some extent, one or more of the symptoms associated with the disease or condition; reduced morbidity and mortality, and improvement in quality of life issues. The above parameters for assessing successful treatment and improvement in the disease or condition are readily measurable by routine procedures familiar to a physician.

The term “thromboinflammation” refers to the uncontrolled development of blood clots with inflammation, worsened by an excessive immune response. Several key components contribute to the development and perpetuation of thromboinflammation, such as platelets, endothelial cells, components of the hemostatic system, immune cells, inflammatory mediators, and ROS. The immune reaction associated to thromboinflammation can occur in response to an injury, triggering an inflammatory response that involves immune cells such as neutrophils, and various inflammatory mediators. Thromboinflammation is thus driven by the interplay between the immune system and the coagulation system, unlike thrombosis which is related to clot formation due to blood flow abnormalities, endothelial damage, or hypercoagulability.

The term “variant” refers to a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions identical. A variant and reference polypeptide may differ in amino acid sequence by one or more substitutions (preferably conservative), additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. Variants should retain one or more of the biological activities of the reference polypeptide.

The term “physiological liquid” is equivalent to the term “saline solution” and designates a solution that is designed to maintain the viability of tissues by containing specific concentrations of substances that are vital for normal tissue function. It can for example be a Ringer's solution or a normal saline solution or any artificial physiological salt solution with specific properties such as a defined pH range, osmotic pressure, and active hydrogen reaction value.

The term “comprised between” associated with a range of value intends to include the extremities of the range.

### DETAILED DESCRIPTION

The present invention thus relates to a stabilized pharmaceutical composition of a protein or polypeptide comprising an Ixodes ricinus salivary gland polypeptide or a fragment or a variant thereof, and its use for inhibiting the intrinsic coagulation pathway and thereby for treating and/or preventing diseases and conditions associated to the intrinsic coagulation pathway, in particular treating and/or preventing thrombus associated to the activation of the intrinsic coagulation pathway.

In some embodiments, the protein or polypeptide comprising an Ixodes ricinus salivary gland polypeptide or a fragment or a variant thereof, comprises or consists of an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 2 or SEQ ID NO: 1. In some embodiments, the protein or polypeptide comprising an Ixodes ricinus salivary gland polypeptide or a fragment or a variant thereof has at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 2.

The present invention relates to a pharmaceutical composition comprising:
a protein or polypeptide comprising or consisting of a polypeptide that has at least 75% sequence identity with the amino acid sequence SEQ ID NO: 2, preferably a protein or polypeptide having the amino acid sequence SEQ ID NO: 2, and
a stabilizing agent of formula (I) to prevent aggregation and/or opalescence

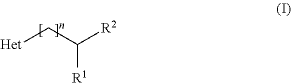

wherein R¹ is H, NH₂ or alanine; R² is H or COOH;
n=0, 1 or 2;
Het is a nitrogen containing heterocycle selected from phenyl, 4-hydroxyphenyl, pyrazol-3-yl, pyrazol-4-yl, pyrazol-5-yl, imidazole-4-yl, 1,2,4-Triazol-3-yl, indol-3-yl, 4-pyridyl, 3-pyridyl, 2-pyridyl.

In certain embodiments, the pharmaceutical composition comprises:
a protein or polypeptide comprising a polypeptide that has at least 75% sequence identity with the amino acid sequence SEQ ID NO: 2, preferably a protein or polypeptide having the amino acid sequence SEQ ID NO: 2, and
a stabilizing agent of formula (I) to prevent aggregation and/or opalescence

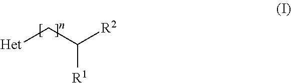

wherein R¹ is H, NH₂ or alanine; R² is H or COOH;
n=0, 1 or 2;
Het is a nitrogen containing heterocycle selected from the group consisting of phenyl, 4-hydroxyphenyl, pyrazol-3-yl, pyrazol-4-yl, pyrazol-5-yl, imidazole-4-yl, 1,2,4-Triazol-3-yl, indol-3-yl, 4-pyridyl, 3-pyridyl, 2-pyridyl,
preferably wherein the stabilizing agent is a compound selected from the group consisting of phenylalanine, tyrosine, histidine, histamine, tryptophane, β-(1,2,4-Triazol-3-yl)-alanine, imidazole-4-acetic acid, Carnosine, imidazolepropionic acid or 4-pyridyl alanine.

The polypeptide was initially prepared and isolated at a concentration of 25 mg/mL in PBS. However, the long-term stability studies on Drug Substance (DS) and Drug Product (DP), led to a recurrent out of specification (OOS) issue concerning clarity and opalescence of samples stored at −20 °C±−5 °C.

The applicant has demonstrated that substituting Phosphate-Buffered Saline (PBS) with a solution comprising a stabilizing agent of Formula (I) allowed to avoid appearance of opalescence and maintain a clear solution.

In a preferred embodiment, the stabilizing agent is a compound selected from phenylalanine, tyrosine, histidine, histamine, tryptophane, β-(1,2,4-Triazol-3-yl)-alanine, imidazole-4-acetic acid, Carnosine, imidazolepropionic acid or 4-pyridyl alanine.

In one embodiment, the stabilizing agent is a compound of formula (II),

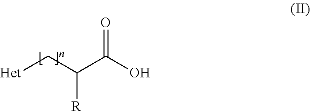

wherein R¹, n and Het are as defined above.

In a preferred embodiment, the stabilizing agent is selected from phenylalanine, tyrosine, histidine, tryptophane, β-(1,2,4-Triazol-3-yl)-alanine, imidazole-4-acetic acid, carnosine, imidazolepropionic acid or 4-pyridyl alanine.

In one embodiment, R¹ is NH₂ and the stabilizing agent is therefore an amino acid. Preferably, the stabilizing agent is an amino acid with L-configuration.

In a preferred embodiment, the stabilizing agent is a compound selected from histidine, tryptophane, β-(1,2,4-Triazol-3-yl)-alanine or 4-pyridyl alanine.

In a preferred embodiment, the stabilizing agent is histidine, preferably L-histidine, i.e. Het is imidazole-4-yl, n=1 and R¹═NH₂ and R²═COOH.

In a preferred embodiment, the stabilizing agent is tryptophane, preferably L-tryptophane, i.e. Het is indol-3-yl, n=1 and R¹═NH₂ and R²═COOH.

In a preferred embodiment, the stabilizing agent is histamine, i.e. Het is imidazole-4-yl, n=1 and R¹═NH₂ and R²═H.

In a preferred embodiment, R¹ is alanine; R² is H or COOH. For example, the stabilizing agent is carnosine, preferably L-Carnosine.

In one embodiment, n=0 or 1. In a preferred embodiment, n=1.

In a preferred embodiment, the pharmaceutical composition of the invention has a concentration of the stabilizing agent comprised between 1 mM and 50 mM, preferably between 10 mM and 40 mM, preferably between 15 mM and 30 mM.

In a preferred embodiment, the pharmaceutical composition of the invention has a pH of 5.0 to 7.5. The pH should indeed be physiologically acceptable. It can be influenced by the pKa of the stabilizing agent. Preferably, the pH is 5.5 to 7.0.

In one embodiment, the pharmaceutical composition is devoid of PBS (Phosphate Buffer Saline).

In a preferred embodiment, the pharmaceutical composition of the invention comprises a protein or polypeptide comprising a polypeptide that has at least 75% sequence identity with the amino acid sequence SEQ ID NO: 2, preferably at least 80% sequence identity, preferably at least 85% identity, preferably at least 90% identity, preferably at least 95% identity with the amino acid sequence SEQ ID NO: 2. In a particular embodiment, the pharmaceutical composition of the invention comprises protein or polypeptide comprising a polypeptide that has the amino acid sequence SEQ ID NO: 2. In a preferred embodiment, in the pharmaceutical composition of the invention, the protein or polypeptide comprising a polypeptide that has at least 75% sequence identity with the amino acid sequence SEQ ID NO: 2 is present at a concentration comprised between 15 mg/mL and 35 mg/mL, preferably between 20 mg/mL and 30 mg/mL, preferably around 25 mg/mL.

It will be understood that the protein or polypeptide comprised in the pharmaceutical composition of the invention is a Ixodes ricinus salivary gland polypeptide. In one embodiment, the Ixodes ricinus salivary gland polypeptide is Ixodes ricinus Contact Phase Inhibitor (Ir—CPI, also referred to as BIOX-101). As used herein, Ir—CPI may also be called IrCPI or BIOX-101. Ir—CPI/BIOX-101 has been previously described by the inventors in, e.g., WO2009141382, WO2019149906, and WO2023222871, incorporated herein by reference in their entirety.

In some embodiments, the Ir—CPI (or BIOX-101) polypeptide comprises or consists of an amino acid sequence having at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity with SEQ ID NO: 2 or SEQ ID NO: 1, preferably SEQ ID NO: 2. In some embodiments, the Ir—CPI (or BIOX-101) polypeptide has an amino sequence comprising or consisting of SEQ ID NO: 1 or SEQ ID NO: 2, preferably SEQ ID NO: 2. In a preferred embodiment, the amino acid sequence of the Ir—CPI (or BIOX-101) polypeptide is at least 75% identical to SEQ ID NO: 2, preferably at least 80%, 85%, 90%, 95%, 96%, 97%, 98%, 99% or 100% identical. In another embodiment, the amino acid sequence of the Ir—CPI (or BIOX-101) polypeptide is at least 75% identical to SEQ ID NO: 1, preferably at least 80%, 85%, 90%, 95%, 96%, 97%, 98%, 99% or 100% identical.

It will be understood that the Ir—CPI (or BIOX-101) polypeptide of SEQ ID NO: 2 has an amino sequence comprising a mutation compared to SEQ ID NO: 1, leading to the replacement of Asparagine in a position corresponding to position 54 in the amino acid sequence of SEQ ID NO: 1 by Glutamine to prevent N-glycosylation.

In one embodiment, fragments of the Ir—CPI (or BIOX-101) polypeptide are also included in the present invention. As used herein, the term “fragment” means a polypeptide having an amino acid sequence that is the same as part, but not all, of the amino acid sequence of the aforementioned Ir—CPI (or BIOX-101) polypeptide. As with the Ir—CPI (or BIOX-101) polypeptide, fragments may be “free-standing” or comprised within a larger polypeptide of which they form a part or region, most preferably as a single continuous region. Preferred fragments include, but are not limited to, truncation polypeptides having the amino acid sequence of the Ir—CPI (or BIOX-101) polypeptide, except for deletion of a continuous series of residues that includes the amino terminus, or a continuous series of residues that includes the carboxyl terminus and/or transmembrane region or deletion of two continuous series of residues, one including the amino terminus and one including the carboxyl terminus. Also preferred are the fragments characterized by structural or functional attributes such as fragments that comprise alpha-helix and alpha-helix forming regions, beta-sheet and beta-sheet forming regions, turn and turn-forming regions, coil and coil-forming regions, hydrophilic regions, hydrophobic regions, alpha amphipathic regions, beta amphipathic regions, flexible regions, surface-forming regions, substrate binding region, and high antigenic index regions. Other preferred fragments are biologically active fragments. Biologically active fragments are those that mediate Ir—CPI (or BIOX-101) polypeptide activity, including those with a similar activity or an improved activity, or with a decreased undesirable activity. In one embodiment, all of these polypeptide fragments retain parts of the biological activity of the Ir—CPI (or BIOX-101) polypeptide.

In one embodiment, variants of the Ir—CPI (or BIOX-101) polypeptide are also included in the present invention. Preferred variants are those that vary from the Ir—CPI (or BIOX-101) polypeptide by conservative amino acid substitutions, i.e., those that substitute a residue with another of like characteristics. Typical substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are variants in which several, 5-10, 1-5, or 1-2 amino acids are substituted, deleted, or added in any combination. Most preferred variants are naturally occurring allelic variants of the Ir—CPI (or BIOX-101) polypeptide present in Ixodes ricinus salivary glands.

In one embodiment, the Ir—CPI (or BIOX-101) polypeptide of the invention can be prepared in any suitable manner. Examples of peptides prepared according to a suitable manner include, without being limited to, isolated naturally occurring polypeptides, recombinantly produced polypeptides, synthetically produced polypeptides, or polypeptides produced by a combination of these methods. Means for preparing such polypeptides are well understood in the art.

In one embodiment, the protein of the invention is a fusion protein comprising Ir—CPI (or BIOX-101).

As used herein, the term “fusion protein” refers to a fusion of the Ir—CPI (or BIOX-101) polypeptide as described hereinabove and at least one other polypeptide.

In one embodiment, the Ir—CPI (or BIOX-101) polypeptide is linked to at least one other polypeptide in such a manner as to produce a single protein which retains the biological activity of Ir—CPI (or BIOX-101). In one embodiment, the polypeptides of the fusion protein are linked C-terminus to N-terminus. In another embodiment, the components of the fusion protein are linked C-terminus to C-terminus. In another embodiment, the components of the fusion protein are linked N-terminus to N-terminus. In another embodiment, the components of the fusion protein are linked N-terminus to C-terminus. In one embodiment, the polypeptides of the fusion protein may be fused in any order. In one embodiment, the polypeptides are disposed in a single, contiguous polypeptide chain.

In one embodiment, the fusion protein of the invention further comprises at least one peptide linker. In one embodiment, the polypeptides of the fusion protein of the invention are linked to each other through one or more peptide linkers.

In one embodiment, the term “Ir—CPI-fusion proteins” or “fusion proteins” indiscriminately refers to fusion proteins without a linker sequence added or with a linker sequence added.

In one embodiment, the linker peptide provides a greater physical separation between the two moieties and thus maximizes the availability of the Ixodes ricinus salivary gland polypeptide. The linker peptide may consist of amino acids such that it is flexible or more rigid.

In one embodiment, the peptide linker of the invention comprises from 1 to 60 amino acids, preferably from 2 to 50 amino acids, more preferably from 4 to 40 amino acids. In one embodiment, the peptide linker of the invention comprises from 5 to 50 amino acids, from 10 to 50 amino acids, from 15 to 50 amino acids, or from 20 to 50 amino acids. In another embodiment, the peptide linker of the invention comprises from 6 to 20 amino acids, from 8 to 20 amino acids, or from 10 to 20 amino acids. In another embodiment, the peptide linker of the invention comprises from 2 to 10 amino acids, from 5 to 20 amino acids, from 10 to 30 amino acids, or from 15 to 40 amino acids.

In one embodiment, the peptide linker of the invention is not an in vivo cleavable linker. In one embodiment, the peptide linker of the invention is an in vivo cleavable linker.

Fusion proteins of the invention may be obtained, for example, by solid-state peptide synthesis (e.g., Merrifield solid phase synthesis) or recombinant production. For recombinant production one or more polynucleotide encoding the fusion protein (fragment), e.g., as described hereinabove, is isolated and inserted into one or more vectors for further cloning and/or expression in a host cell. Such polynucleotide may be readily isolated and sequenced using conventional procedures.

Methods which are well known to those skilled in the art can be used to construct expression vectors containing the coding sequence of a fusion protein (fragment) along with appropriate transcriptional/translational control signals. These methods include in vitro recombinant DNA techniques, synthetic techniques and in vivo recombination/genetic recombination. The expression vector can be part of a plasmid, virus, or may be a nucleic acid fragment. The expression vector includes an expression cassette into which the polynucleotide encoding the fusion protein (fragment) (i.e., the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g., on a single vector, or in separate polynucleotide constructs, e.g., on separate (different) vectors. Furthermore, any vector may contain a single coding region, or may comprise two or more coding regions, e.g., a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the fusion protein (fragment) of the invention, or variant or derivative thereof. Heterologous coding regions include without limitation specialized elements or motifs, such as a secretory signal peptide or a heterologous functional domain. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g., the immediate early promoter, in conjunction with intron-A), simian virus 40 (e.g., the early promoter), and retroviruses (such as, e.g., Rous sarcoma virus). Other transcription control regions include those derived from vertebrate genes such as actin, heat shock protein, bovine growth hormone and rabbit a-globin, as well as other sequences capable of controlling gene expression in eukaryotic cells. Additional suitable transcription control regions include tissue-specific promoters and enhancers as well as inducible promoters (e.g., promoters inducible tetracyclins). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Fusion proteins prepared as described herein may be purified by art-known techniques such as high-performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the fusion protein binds. The purity of the fusion protein can be determined by any of a variety of well-known analytical methods including gel electrophoresis, high pressure liquid chromatography, and the like.

In some embodiments, the protein or polypeptide has the amino acid sequence SEQ ID NO: 2; and the stabilizing agent is histidine, preferably L-histidine.

Thus, in some embodiments, the pharmaceutical composition of the invention comprises or consists of:
a polypeptide or protein that has at least 75% sequence identity with the amino acid sequence SEQ ID NO: 2, preferably a polypeptide or protein having the amino acid sequence SEQ ID NO: 2, and
a stabilizing agent to prevent aggregation and/or opalescence being histidine, preferably L-histidine.

In some embodiments, the pharmaceutical composition of the invention comprises or consists of (i) a polypeptide having the amino acid sequence SEQ ID NO: 2 and (ii) histidine, preferably L-histidine.

The pharmaceutical composition of the invention can be diluted for intravenous administration to patients. The invention therefore also relates to an injectable solution comprising the pharmaceutical composition as defined above diluted with a physiological liquid or saline solution.

In a preferred embodiment, the injectable solution comprises
a polypeptide that has at least 75% sequence identity with the amino acid sequence SEQ ID NO: 2, and
a stabilizing agent of formula (I) to prevent aggregation and/or opalescence

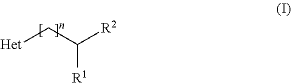

wherein R¹, R², n and Het are as described above, and
a physiological liquid or saline solution.

In one embodiment, in the injectable solution of the invention, the protein or polypeptide comprising a polypeptide that has at least 75% sequence identity with the amino acid sequence SEQ ID NO: 2 is present at a concentration comprised between 2 mg/mL and 10 mg/mL, preferably between 3 mg/mL and 7.5 mg/mL, preferably between 4 mg/mL and 6 mg/mL, preferably around 5 mg/mL.

In one embodiment, the pharmaceutical composition of the invention is provided as a kit for reconstitution of an injectable solution. The invention therefore also relates to a kit comprising:
the pharmaceutical composition as defined above and
a physiological liquid or saline solution.

In one embodiment, the kit of the invention further comprises means to administer the pharmaceutical composition and physiological liquid to a subject in need thereof.

In one embodiment, the kit of the invention further comprises instructions for the administration of the pharmaceutical composition and physiological liquid to said subject.

In a preferred embodiment, the kit comprises a volume of physiological liquid sufficient to dilute the pharmaceutical composition to an injectable concentration of protein or polypeptide. For example, the kit comprises a volume of physiological liquid 1.5 to 17.5 times higher, 2 to 15 times, 4 to 10 times higher or 5 times higher than the volume of pharmaceutical composition.

The invention also relates to the use of a stabilizing agent as defined above for stabilizing a protein or polypeptide comprising a polypeptide that has at least 75% sequence identity with the amino acid sequence SEQ ID NO: 2.

In a preferred embodiment, the invention relates to the use of histidine, and in particular L-histidine, for stabilizing a protein or polypeptide comprising a polypeptide that has at least 75% sequence identity with the amino acid sequence SEQ ID NO: 2 and preferably for stabilizing the polypeptide that has the amino acid sequence SEQ ID NO:

2.

Medicals uses of Ir—CPI/BIOX-101 have been previously described by the inventors in, e.g., WO2009141382, WO2019149906, and WO2023222871, incorporated herein by reference in their entirety.

The invention further relates to the medical use of the pharmaceutical composition, the injectable solution or the kit as defined above. The invention thus further relates to the pharmaceutical composition, the injectable solution or the kit as defined above, for use as a medicament.

The present invention further relates to the pharmaceutical composition, injectable solution, or the kit as described herein, for use for preventing and/or treating a disease, preferably a disease selected from the group consisting of:
thrombotic disorders, preferably selected from the group consisting of venous thrombosis such as deep vein thrombosis, pulmonary embolism, renal vein thrombosis, and hepatic vein thrombosis; arterial thrombosis such as myocardial infarction and peripheral artery disease; and thromboembolism;
cerebrovascular diseases, preferably selected from the group consisting of ischemic stroke, hemorrhagic stroke such as intracerebral hemorrhage (ICH) and subarachnoid hemorrhage (SAH), thrombus formation following cerebral injuries, and cerebral ischemia;
atherosclerosis;
conditions associated with thrombosis such as preeclampsia, eclampsia, and HELLP syndrome;
inflammatory disorders associated with thrombosis such as rheumatoid arthritis, sepsis, and inflammatory bowel disease;
thromboinflammation;
brain inflammation, preferably brain inflammation occurring after a brain injury and/or a brain hemorrhage such as intracerebral hemorrhage (ICH), more preferably wherein the pharmaceutical composition, injectable solution, or the kit as described herein is administered within 120 hours, 96 hours or 72 hours after the onset of brain injury and/or brain hemorrhage; and
neuronal cell death, preferably neuronal cell death occurring after a brain injury and/or a brain hemorrhage such as intracerebral hemorrhage (ICH), more preferably wherein the pharmaceutical composition, injectable solution, or the kit as described herein is administered within 120 hours, 96 hours or 72 hours after the onset of brain injury and/or brain hemorrhage.

The present invention further relates to the pharmaceutical composition, injectable solution, or the kit as described herein, for use for preventing and/or treating a disease, preferably a disease selected from the group consisting of:
a thrombotic disorder,
a cerebrovascular disease,
inflammatory disorders associated with thrombosis,
thromboinflammation,
conditions associated with thrombosis,
a disease associated with platelet recruitment, neutrophil recruitment, neutrophil activation and/or NETosis,
brain inflammation, preferably brain inflammation occurring after a brain injury and/or a brain hemorrhage, more preferably wherein the pharmaceutical composition, injectable solution, or the kit as described herein is administered within 120 hours, 96 hours or 72 hours after the onset of brain injury and/or brain hemorrhage, and
neuronal cell death, preferably neuronal cell death occurring after a brain injury and/or a brain hemorrhage, more preferably wherein the pharmaceutical composition, injectable solution, or the kit as described herein is administered within 120 hours, 96 hours or 72 hours after the onset of brain injury and/or brain hemorrhage.

The present invention further relates to the pharmaceutical composition, injectable solution, or the kit as described herein, for use for preventing and/or treating a disease, preferably a disease selected from the group consisting of:
thrombotic disorders, preferably selected from the group consisting of venous thrombosis such as deep vein thrombosis, pulmonary embolism, renal vein thrombosis, and hepatic vein thrombosis; arterial thrombosis such as myocardial infarction and peripheral artery disease; and thromboembolism;
cerebrovascular diseases, preferably selected from the group consisting of ischemic stroke, hemorrhagic stroke such as intracerebral hemorrhage (ICH) and subarachnoid hemorrhage (SAH), thrombus formation following cerebral injuries, and cerebral ischemia;
atherosclerosis;
conditions associated with thrombosis such as preeclampsia, eclampsia, and HELLP syndrome; and
inflammatory disorders associated with thrombosis such as rheumatoid arthritis, sepsis, and inflammatory bowel disease;
   preferably the disease is stroke.

In some embodiments, the pharmaceutical composition, injectable solution, or the kit as described herein, is for use for preventing and/or treating a disease selected from the group consisting of thrombotic disorders, cerebrovascular disease, ischemic stroke, hemorrhagic stroke, cerebral ischemia, atherosclerosis, thrombus formation following cerebral injuries, thrombosis associated with preeclampsia, sepsis, rheumatoid arthritis, inflammatory bowel disease, thromboembolism, myocardial infarction, pulmonary embolism, renal vein thrombosis and hepatic vein thrombosis.

In some embodiments, the pharmaceutical composition, injectable solution, or the kit as described herein, is for use for preventing and/or treating a disease selected from the group consisting of thrombotic disorder, thromboinflammation and brain inflammation. In some embodiments, the pharmaceutical composition, injectable solution, or the kit as described herein, is for use for preventing and/or treating a disease selected from the group consisting of stroke, thromboinflammation and brain inflammation. In some embodiments, the pharmaceutical composition, injectable solution, or the kit as described herein, is for use for preventing and/or treating a disease selected from the group consisting of thrombotic disorder, thromboinflammation and brain inflammation occurring or susceptible to occur after a brain hemorrhage and/or a brain injury. In some embodiments, the pharmaceutical composition, injectable solution, or the kit as described herein, is for use for preventing and/or treating a disease selected from the group consisting of thromboinflammation and brain inflammation occurring or susceptible to occur after a brain hemorrhage and/or a brain injury.

In some embodiments, the disease is a thrombotic disorder.

In some embodiments, the thrombotic disorder is selected from the group consisting of venous thrombosis, arterial thrombosis, and thromboembolism. Non limitative examples of venous thrombosis include deep vein thrombosis, pulmonary embolism, renal vein thrombosis, and hepatic vein thrombosis. Non limitative examples of arterial thrombosis include myocardial infarction and peripheral artery disease.

In one embodiment, the thrombotic disorder is selected from the group consisting of deep vein thrombosis, pulmonary embolism, renal vein thrombosis, and hepatic vein thrombosis. In another embodiment, the thrombotic disorder is selected from the group consisting of myocardial infarction and peripheral artery disease. In another embodiment, the thrombotic disorder is thromboembolism.

In some embodiments, the disease is a cerebrovascular disease.

In some embodiments, the cerebrovascular disease is selected from the group consisting of ischemic stroke, hemorrhagic stroke such as intracerebral hemorrhage (ICH) and subarachnoid hemorrhage (SAH), thrombus formation following cerebral injuries, and cerebral ischemia. In certain embodiments, the cerebrovascular disease selected is from the group consisting of stroke, intracranial stenosis, and intracranial aneurysms.

In some embodiments, the disease is stroke. In one embodiment, the disease is ischemic stroke. In another embodiment, the disease is hemorrhagic stroke, preferably selected from the group consisting of intracerebral hemorrhage (ICH) and subarachnoid hemorrhage (SAH), more preferably the hemorrhagic stroke is intracerebral hemorrhage.

In one embodiment, the disease is intracerebral hemorrhage (ICH). In another embodiment, the disease is subarachnoid hemorrhage (SAH).

As used herein, “ICH” refers to bleeding into the brain parenchyma, and “SAH” refers to bleeding into the subarachnoid space. ICH induces (1) primary brain injury through direct mechanical effects of the hemorrhage, and (2) secondary brain injury (SBI) comprising inter alia the disruption of the blood-brain-barrier (BBB), perihematomal edema (PHE), death of brain parenchymal cells, neuroinflammation, and neuronal cell death.

In some embodiments, the disease is a condition associated with thrombosis.

In some embodiments, the condition associated with thrombosis is selected from the group consisting of preeclampsia, eclampsia, and HELLP syndrome.

In some embodiments, the disease is an inflammatory disorder associated with thrombosis.

In some embodiments, the inflammatory disorder associated with thrombosis is selected from the group consisting of rheumatoid arthritis, sepsis, and inflammatory bowel disease.

In some embodiments, the disease is selected from the group consisting of stroke, thromboembolism, myocardial infarction, cerebrovascular diseases, cerebral ischemia, pulmonary embolism, and thrombosis such as renal vein thrombosis and hepatic vein thrombosis.

In some embodiments, the disease is atherosclerosis.

The present invention also relates to the pharmaceutical composition, injectable solution, or the kit as described herein, for use for preventing and/or treating a disease or condition associated with platelet recruitment, neutrophil recruitment, neutrophil activation and/or NETosis, preferably the disease or condition associated with platelet recruitment, neutrophil recruitment, neutrophil activation and/or NETosis.

In some embodiments, the disease is a disease or condition associated with platelet recruitment, neutrophil recruitment, neutrophil activation and/or NETosis, preferably the disease or condition associated with platelet recruitment, neutrophil recruitment, neutrophil activation and/or NETosis is thromboinflammation or inflammatory diseases with thrombotic tendency, preferably thromboinflammation. In some embodiments, the disease is thromboinflammation, thus the present invention also relates to the pharmaceutical composition, injectable solution, or the kit as described herein, for use for preventing and/or treating thromboinflammation. In another embodiment, the disease is inflammatory diseases with thrombotic tendency.

As used herein, the term “thromboinflammation” refers to the uncontrolled development of blood clots with inflammation, worsened by an excessive immune response. Several key components contribute to the development and perpetuation of thromboinflammation, such as platelets, endothelial cells, components of the hemostatic system, immune cells, inflammatory mediators, and ROS. The immune reaction associated to thromboinflammation can occur in response to an injury, triggering an inflammatory response that involve immune cells such as neutrophils, and various inflammatory mediators. Thromboinflammation is thus driven by the interplay between the immune system and the coagulation system, unlike thrombosis which is related to clot formation due to blood flow abnormalities, endothelial damage, or hypercoagulability.

As used herein, the term “inflammatory diseases with thrombotic tendency” may be replaced by “thrombotic complications in inflammatory diseases”.

In some embodiments, the disease is thromboinflammation selected from the group consisting of atherosclerosis-induced thromboinflammation, plaque rupture-induced thromboinflammation, devices-induced thromboinflammation, catheterism-induced and/or stent-induced thromboinflammation, extracorporeal circulation-induced thromboinflammation, cerebral injuries-induced thromboinflammation, coronary artery disease-induced thromboinflammation, acute myocardial infraction-induced thromboinflammation, cancer-induced thromboinflammation, metastasis-induced thromboinflammation, stroke-induced thromboinflammation, Behçet's disease (BD)-induced thromboinflammation, antineutrophil cytoplasmic antibody-associated (ANCA) thromboinflammation, Takayasu arteritis-induced vasculitides-induced thromboinflammation, rheumatoid arthritis-induced thromboinflammation, systemic lupus erythematosus-induced thromboinflammation, antiphospholipid syndrome-induced thromboinflammation, familial Mediterranean fever-induced thromboinflammation, thromboangiitis obliterans (TAO)-induced thromboinflammation, sepsis-induced thromboinflammation, inflammatory bowel diseases-induced thromboinflammation, heparin-induced thrombocytopenia-induced thromboinflammation, immunothrombosis-induced thromboinflammation, (pre) eclampsia-induced thromboinflammation, HELLP syndrome-induced thromboinflammation, thromboinflammation complication in cell and cell cluster transplantation and in whole organ transplantation or grafts, venous thromboembolism-induced thromboinflammation, aneurysms-induced thromboinflammation, and skeletal muscle ischemia-reperfusion syndrome-induced thromboinflammation.

In some embodiments, the disease is thromboinflammation selected from the group consisting of cerebral injuries-induced thromboinflammation, cancer-induced thromboinflammation, metastasis-induced thromboinflammation, aneurysms-induced thromboinflammation, and stroke-induced thromboinflammation. In some embodiments, the disease is thromboinflammation selected from the group consisting of cerebral injuries-induced thromboinflammation, aneurysms-induced thromboinflammation, and stroke-induced thromboinflammation. In some embodiments, the disease is thromboinflammation selected from the group consisting of cerebral injuries-induced thromboinflammation, and stroke-induced thromboinflammation.

In some embodiments, the disease is cerebral injuries-induced thromboinflammation.

In some embodiments, the disease is stroke-induced thromboinflammation.

In another embodiment, the disease is selected from the group comprising or consisting of thrombosis (including venous and arterial thrombosis), diseases and conditions associated to cardiac surgical interventions, cardiovascular diseases, cancer and metastasis.

In some embodiments, the disease is brain inflammation. In some embodiments, the disease is brain inflammation occurring or susceptible to occur after brain hemorrhage. In some embodiments, the disease is brain inflammation occurring or susceptible to occur after brain injury. Thus, the present invention also relates to the pharmaceutical composition, injectable solution, or the kit as described herein, for use for preventing and/or treating brain inflammation, preferably brain inflammation occurring or susceptible to occur after brain injury, more preferably brain inflammation occurring or susceptible to occur after brain hemorrhage. The present invention also relates to the pharmaceutical composition, injectable solution, or the kit as described herein, for use for preventing and/or treating neuronal cell death, preferably neuronal cell death occurring or susceptible to occur after brain injury, more preferably neuronal cell death occurring or susceptible to occur after brain hemorrhage.

In a preferred embodiment, the brain hemorrhage is intracerebral hemorrhage (ICH) or subarachnoid hemorrhage (SAH). In a more preferred embodiment, the brain hemorrhage is intracerebral hemorrhage (ICH).

In some embodiments, the pharmaceutical composition, injectable solution, or the kit for use according to the invention prevents and/or treats brain inflammation. In some embodiments, the pharmaceutical composition, injectable solution, or the kit for use according to the invention prevents or decreases the likelihood of brain inflammation. In some embodiments, the pharmaceutical composition, injectable solution, or the kit for use according to the invention treats brain inflammation.

As used herein the term “brain inflammation”, used interchangeably with the term “neuroinflammation”, refers to the onset of inflammation in the central nervous system and may involve, inter alia, the activation of glial cells and the secretion of pro-inflammatory cytokines. The process of neuroinflammation affects neurons, microglia and macroglia.

In some embodiments, the brain inflammation comprises or consists of the release of proinflammatory mediators in the brain. Non limitative examples of proinflammatory cytokines include interleukins (IL) such as IL-1, IL-2, IL-12, IL-17 and IL-18; IFN-γ; and TNF-α.

In some embodiments, the brain inflammation comprises or consists of the early activation of resident microglia. As used herein, “microglia” refers to the resident macrophages of the central nervous system.

In some embodiments, the brain inflammation comprises or consists of the formation of reactive oxygen species (ROS). Non-limitative examples of ROS include superoxide, hydroxyl radical, singlet oxygen, nitrous oxide and peroxides (e.g., hydrogen peroxide). In some embodiments, the formation of ROS induces oxidative stress.

In some embodiments, the brain inflammation comprises or consists of infiltration of systemic inflammatory cells in the brain. In some embodiments, the systemic inflammatory cells are selected from the group comprising or consisting of neutrophils, eosinophils, lymphocytes, plasma cells, and histiocytes. In some embodiments, the systemic inflammatory cells are neutrophils.

In some embodiments, the brain inflammation is associated or induces excitotoxicity. As used herein, the term “excitotoxicity” refers to a state of abnormal stimulation of neurotransmitters receptors in a neuron, leading inter alia to altered calcium and energy homeostasis and apoptosis.

In some embodiments, the brain inflammation is associated with or induces neuronal cell death.

In one embodiment, the brain inflammation is localized to a specific region or area of the brain. Typically, the brain inflammation is localized at the site and/or at the periphery of a brain injury, e.g., a brain hemorrhage such as a intracerebral hemorrhage (ICH) or subarachnoid hemorrhage (SAH).

In another embodiment, the brain inflammation is not localized to a specific region or area of the brain. In certain embodiments, the brain inflammation affects all the brain.

In some embodiments, the brain inflammation is not associated with the presence or the formation of a thrombus. In some embodiments, the brain inflammation is not induced by the presence or the formation of a thrombus. In some embodiments, the brain inflammation does not lead to the presence or the formation of a thrombus. In certain embodiments, the pharmaceutical composition, injectable solution, or the kit described herein above is not used for preventing and/or treating thrombotic-related or thrombotic-induced diseases.

In some embodiments, the brain inflammation is an induced brain inflammation. In some embodiments, the induced brain inflammation is due to a brain injury. In some embodiments, the induced brain inflammation is due to a traumatic brain injury. In some embodiments, the brain inflammation is induced by a brain hemorrhage, preferably an intracerebral hemorrhage (ICH) or a subarachnoid hemorrhage (SAH), more preferably an ICH.

In some embodiments, the brain inflammation is due to a brain injury. In some embodiments, the brain injury is a traumatic brain injury. In some other embodiments, the brain injury is a non-traumatic brain injury.

In some embodiments, the brain inflammation is due to an acquired brain injury. In some embodiments, the acquired brain injury is a traumatic brain injury. In some other embodiments, the acquired brain injury is a non-traumatic brain injury.

In some embodiments, the traumatic brain injury is selected from the group comprising or consisting of hemorrhage, contusion, concussion, and penetrating brain injury. In some embodiments, the traumatic brain injury is hemorrhage, preferably an intracerebral hemorrhage (ICH) or a subarachnoid hemorrhage (SAH), more preferably an ICH.

In some embodiments, the non-traumatic brain injury is selected from the group comprising or consisting of brain hemorrhage, ischemic stroke, seizure, infection, brain tumor, hypoxic/anoxic brain injury, autoimmune disease, toxicity and hydrocephalus. In some embodiments, the non-traumatic brain injury is selected from the group comprising or consisting of brain hemorrhage, seizure, infection, brain tumor, hypoxic/anoxic brain injury, autoimmune disease, toxicity and hydrocephalus. In some embodiments, the non-traumatic brain injury is selected from the group comprising or consisting of brain hemorrhage, seizure, infection, hypoxic/anoxic brain injury, autoimmune disease, toxicity and hydrocephalus. In some embodiments, the non-traumatic brain injury is selected from the group comprising or consisting of brain hemorrhage, infection, brain tumor, hypoxic/anoxic brain injury, autoimmune disease, toxicity and hydrocephalus. In some embodiments, the non-traumatic brain injury is selected from the group comprising or consisting of brain hemorrhage, infection, hypoxic/anoxic brain injury, autoimmune disease, toxicity and hydrocephalus. In some embodiments, the non-traumatic brain injury is brain hemorrhage. In some embodiments, the non-traumatic brain injury is autoimmune disease.

In some embodiments, the infection is selected from the group comprising or consisting of viral infection, bacterial infection, fungal infection, parasitic infection, and combinations thereof. In some embodiments, the infection is induced by a septicemia and/or a sepsis. In some embodiments, the infection is a viral infection. In some embodiments, the infection is a bacterial infection. In some embodiments, the infection is a fungal infection. In some embodiments, the infection is a parasitic infection.

In some embodiments, the brain injury is selected from the group comprising or consisting of brain hemorrhage, contusion, concussion, penetrating brain injury, ischemic stroke, seizure, infection, brain tumor, hypoxic/anoxic brain injury, autoimmune disease, toxicity and hydrocephalus. In some embodiments, the brain injury is selected from the group comprising or consisting of brain hemorrhage, contusion, concussion, penetrating brain injury, seizure, infection, brain tumor, hypoxic/anoxic brain injury, autoimmune disease, toxicity and hydrocephalus. In some embodiments, the brain injury is selected from the group comprising or consisting of brain hemorrhage, contusion, concussion, penetrating brain injury, infection, brain tumor, hypoxic/anoxic brain injury, autoimmune disease, toxicity and hydrocephalus. In some embodiments, the brain injury is selected from the group comprising or consisting of brain hemorrhage, contusion, concussion, penetrating brain injury, seizure, infection, hypoxic/anoxic brain injury, autoimmune disease, toxicity and hydrocephalus.

In some embodiments, the brain inflammation occurred or is susceptible to occur after a traumatic brain injury. In some embodiments, the brain inflammation occurred or is susceptible to occur after a brain hemorrhage. In some embodiments, the pharmaceutical composition, injectable solution, or the kit according to the invention is for use for preventing and/or treating brain inflammation following brain hemorrhage in a subject in need thereof.

In a specific embodiment, the pharmaceutical composition, injectable solution, or the kit of the invention is for use for preventing brain inflammation that is susceptible to occur after a brain injury in a subject in need thereof. In a specific embodiment, the pharmaceutical composition, injectable solution, or the kit of the invention is for use for preventing brain inflammation that is susceptible to occur after a brain hemorrhage in a subject in need thereof.

In some embodiments, the brain inflammation is an acquired brain inflammation or a spontaneous brain inflammation.

In some embodiments, the brain inflammation is an acquired brain inflammation.

As used herein, the term “acquired brain inflammation” refers to a brain inflammation as described hereinabove wherein the cause triggering the inflammation is external to the organism, e.g., a mechanical shock to the head, an object penetrating the skull, or an infection.

In some embodiments, the acquired brain inflammation is induced by a traumatic brain injury or an infection.

In some embodiments, the acquired brain inflammation is induced by a traumatic brain injury.

In some embodiments the traumatic brain injury is selected from the group comprising or consisting of hemorrhage, contusion, concussion, penetrating brain injury, and anoxic brain injury. In some embodiments, the traumatic brain injury is selected from the group comprising or consisting of hemorrhage, contusion, concussion, and penetrating brain injury.

In some embodiments the traumatic brain injury is a hemorrhage selected from the group comprising or consisting of intracerebral hemorrhage, subarachnoid hemorrhage, subdural hemorrhage and epidural hemorrhage. In some embodiments the primary brain injury is an intracerebral hemorrhage. In some embodiments the primary brain injury is a subarachnoid hemorrhage. In some embodiments the primary brain injury is a subdural hemorrhage. In some embodiments the primary brain injury is an epidural hemorrhage.

In some embodiments the traumatic brain injury is a contusion. In some embodiments the traumatic brain injury is a concussion. In some embodiments the traumatic brain injury is a penetrating brain injury. In some embodiments the traumatic brain injury is an anoxic brain injury.

In some embodiments, the acquired brain inflammation is induced by an infection. In some embodiments, the acquired brain inflammation is induced by an infection selected from the group comprising or consisting of viral infection, bacterial infection, fungal infection, parasitic infection, and combinations thereof.

In some embodiments, the acquired brain inflammation is induced by a septicemia and/or a sepsis.

In some embodiments, the acquired brain inflammation is induced by a viral infection. Non-limitative examples of viral infections enabling brain inflammation comprise infections by herpes simplex virus, Epstein-Barr virus, cytomegalovirus, varicella virus, poliovirus, influenza virus, SARS-COV-2, West Nile virus, enterovirus, Chikungunya virus and Dengue virus.

In some embodiments, the acquired brain inflammation is induced by a bacterial infection. Non-limitative examples of bacterial infections enabling brain inflammation comprise infections by bacteria of the genus Treponema, Mycoplasma, Neisseria, Streptococcus and Listeria.

In some embodiments, the acquired brain inflammation is induced by a fungal infection. Non-limitative examples of fungal infections enabling brain inflammation comprise infections by fungi of the genus Candida, Cladosporium, Cryptococcus, Histoplasma and Aspergillus.

In some embodiments, the acquired brain inflammation is induced by a parasitic infection. Non-limitative examples of parasitic infections enabling brain inflammation comprise infections by parasites of the genus Plasmodium and Toxoplasma.

In some embodiments, the brain inflammation is a spontaneous brain inflammation.

As used herein, the term “spontaneous brain inflammation” refers to a brain inflammation as described hereinabove wherein the cause triggering the inflammation is intrinsic to the organism and results, e.g., from an autonomous disease or condition.

In some embodiments, the spontaneous brain inflammation is induced by a non-traumatic brain injury or an autoimmune disease.

In some embodiments, the spontaneous brain inflammation is induced by a non-traumatic brain injury. In certain embodiments, the non-traumatic brain injury is selected from the group comprising or consisting of spontaneous hemorrhage, brain cancer and stroke.

In some embodiments the non-traumatic brain injury is a non-traumatic spontaneous hemorrhage. In certain embodiments, the non-traumatic spontaneous hemorrhage is a non-traumatic spontaneous intracerebral hemorrhage or a spontaneous subarachnoid hemorrhage. In certain embodiments, the non-traumatic spontaneous hemorrhage is a non-traumatic spontaneous intracerebral hemorrhage. In certain embodiments, the non-traumatic spontaneous hemorrhage is a spontaneous subarachnoid hemorrhage.

In some embodiments the non-traumatic brain injury is a brain cancer, e.g., glioblastoma, oligodendroglioma and meningioma.

In some embodiments the non-traumatic brain injury is a stroke. In some embodiments the non-traumatic brain injury is an ischemic stroke.

In some embodiments, the spontaneous brain inflammation is induced by an autoimmune disease. In some embodiments, the autoimmune disease is autoimmune encephalitis. In some embodiments, the non-traumatic brain injury is induced by an autoimmune disease. In some embodiments, the autoimmune disease is autoimmune encephalitis.

Brain inflammation may be associated with, or induced by, other conditions or symptoms such as, e.g., neuronal cell death, excitotoxicity, microglia activation, oxidative stress and infiltration of the brain by systemic inflammatory cells such as neutrophils.

In some embodiments, preventing and/or treating brain inflammation using the pharmaceutical composition, injectable solution, or the kit according to the invention further treats and/or alleviates another disease, condition or symptom. In some embodiments, the disease, condition or symptom is associated with brain inflammation. In some embodiments, the disease, condition or symptom is induced by brain inflammation.

In some embodiments, the pharmaceutical composition, injectable solution, or the kit further prevents and/or treats a disease, condition or symptom associated with brain inflammation. In some embodiments, the pharmaceutical composition, injectable solution, or the kit further prevents and/or treats a disease, condition or symptom induced by brain inflammation.

In some embodiments, preventing and/or treating brain inflammation using the pharmaceutical composition, injectable solution, or the kit according to the invention has a positive effect on another disease, condition or symptom selected from the group comprising or consisting of neuronal cell death, excitotoxicity, microglia activation, oxidative stress and infiltration of the brain by systemic inflammatory cells. In some embodiments, the disease, condition or symptom is selected from the group comprising or consisting of neuronal cell death, excitotoxicity, microglia activation, and oxidative stress. As used herein, the expression “positive effect” is intended to mean that the pharmaceutical composition, injectable solution, or the kit according to the invention alleviates, decreases or suppresses a symptom, or treats partially or entirely a component of the disease or condition.

In some embodiments, preventing and/or treating brain inflammation using the pharmaceutical composition, injectable solution, or the kit according to the invention has a positive effect on neuronal cell death. In some embodiments, preventing and/or treating brain inflammation using the pharmaceutical composition, injectable solution, or the kit according to the invention has a positive effect on excitotoxicity.

In some embodiments, preventing and/or treating brain inflammation using the pharmaceutical composition, injectable solution, or the kit according to the invention has a positive effect on microglia activation.

In some embodiments, preventing and/or treating brain inflammation using the pharmaceutical composition, injectable solution, or the kit according to the invention has a positive effect on oxidative stress. In some embodiments, preventing and/or treating brain inflammation using the pharmaceutical composition, injectable solution, or the kit according to the invention inhibits the formation of reactive oxygen species.

In some embodiments, preventing and/or treating brain inflammation using the pharmaceutical composition, injectable solution, or the kit according to the invention has a positive effect on the infiltration of the brain by systemic inflammatory cells. In certain embodiments the systemic inflammatory cells are selected from the group comprising or consisting of neutrophils, eosinophils, lymphocytes, plasma cells, and histiocytes. In certain embodiments the systemic inflammatory cells are neutrophils. In some embodiments, preventing and/or treating brain inflammation using the pharmaceutical composition, injectable solution, or the kit according to the invention inhibits the recruitment of neutrophils to the brain and/or the formation of neutrophils extracellular traps in the brain.

In some embodiments, the pharmaceutical composition, injectable solution, or the kit for use according to the invention is for preventing and/or treating the long-term effects of a brain injury. As used herein, the expression “long-term effects of a brain injury” refers to the pathophysiological consequences of a brain injury, i.e., the damages impacting at least one cell type in the brain, preferably neurons, for a prolonged period of time, typically one week, one month, one year or more. In some embodiment, the pharmaceutical composition, injectable solution, or the kit for use according to the invention is for preventing and/or treating the long-term effects of a non-traumatic brain injury. In some embodiment, the pharmaceutical composition, injectable solution, or the kit for use according to the invention is for preventing and/or treating the long-term effects of a traumatic brain injury. In some embodiments, the pharmaceutical composition, injectable solution, or the kit for use according to the invention is for preventing and/or treating the long-term effects of a traumatic brain injury associated to hemorrhage. In some embodiments, the pharmaceutical composition, injectable solution, or the kit for use according to the invention is for preventing and/or treating the long-term effects of a brain injury associated to hemorrhage. In some embodiment, the pharmaceutical composition, injectable solution, or the kit for use according to the invention is for preventing and/or treating the long-term effects of a brain hemorrhage.

The Inventors have demonstrated that the administration of the pharmaceutical composition, injectable solution, or the kit of the invention does not increase the hemorrhagic volume nor the lesion (oedema) volume. This property is advantageous while being surprising when one knows the anti-thrombotic effects of this protein.

In certain embodiments, the pharmaceutical composition, injectable solution, or the kit of the invention is for use in preventing and/or treating brain inflammation without increasing hemorrhagic and/or lesion (oedema) volumes. In certain embodiments, the brain inflammation is due to or induced by a cerebral hemorrhage. In one embodiment, the pharmaceutical composition, injectable solution, or the kit of the invention is for use in preventing and/or treating cerebral injury after cerebral hemorrhage. In one embodiment, cerebral injury arises after hemorrhagic stroke. In one embodiment, the pharmaceutical composition, injectable solution, or the kit of the invention is for use in preventing and/or treating long-term effect brain injury after cerebral hemorrhage.

In some embodiments, the pharmaceutical composition, injectable solution, or the kit as described herein, is administered within about 120 hours after the onset of brain inflammation, preferably within 96 hours, more preferably within 72 hours. In some embodiments, the pharmaceutical composition, injectable solution, or the kit as described herein, is administered within about 120 hours after the onset of brain hemorrhage, preferably within 96 hours, more preferably within 72 hours. In some embodiments, the pharmaceutical composition, injectable solution, or the kit as described herein, is administered within about 120 hours after the onset of brain injury, preferably within 96 hours, more preferably within 72 hours.

In some embodiments, the disease is neuronal cell death. In some embodiments, the disease is neuronal cell death occurring or susceptible to occur after brain hemorrhage. In some embodiments, the disease is neuronal cell death occurring or susceptible to occur after brain injury.

In some embodiments, the pharmaceutical composition, injectable solution, or the kit as described herein, is administered within about 120 hours after the onset of brain inflammation, preferably within 96 hours, more preferably within 72 hours. In some embodiments, the pharmaceutical composition, injectable solution, or the kit as described herein, is administered within about 120 hours after the onset of brain hemorrhage, preferably within 96 hours, more preferably within 72 hours. In some embodiments, the pharmaceutical composition, injectable solution, or the kit as described herein, is administered within about 120 hours after the onset of brain injury, preferably within 96 hours, more preferably within 72 hours.

The Inventors have shown the possibility and the safety of administering the protein of the invention rapidly after the appearance of the brain inflammation, brain injury or brain hemorrhage. It is particularly beneficial for patients with brain inflammation, brain injury or brain hemorrhage but susceptible to thrombotic events, such as for example bedridden patients. Indeed, during the first 72 hours after the onset of hemorrhage, anticoagulants (such as enoxaparin) cannot be given because they increase the risk of bleeding. The Inventors have demonstrated that the risk of bleeding is not increased with the protein of the invention during the 3 days after the onset of hemorrhage, unlike enoxaparin (see Example 1). It is therefore possible to treat brain inflammation and/or brain injury with the protein of the invention within a crucial time window to avoid irreversible effects. For best results, the therapeutic solution to brain inflammation must be provided before the inflammatory reaction sets in after the onset of bleeding. Current solutions cannot be administered as quickly because of their increased risk of bleeding. The protein or polypeptide of the invention does not have this disadvantage, on the contrary, and can therefore be administered quickly to reduce the inflammatory reaction before it leads to too harmful and/or permanent effects.

Moreover, the peak of neutrophil infiltration occurs within this 72-hour window, as well as the rapid growth phase of the edema if any. The sooner the size of the edema is reduced, the better the patient outcome.

Therefore, in some embodiments, the pharmaceutical composition, the injectable solution or the kit as defined above is administered as soon as possible after the onset of brain inflammation and/or after brain injury. In some embodiments, the pharmaceutical composition, the injectable solution or the kit as defined above is administrated to the subject rapidly after being exposed to a risk to develop brain inflammation. In some embodiments, the pharmaceutical composition, the injectable solution or the kit as defined above is administered within 120 hours from the onset of brain inflammation and/or after a brain injury, preferably within 96 hours, more preferably within 72 hours. In some embodiments, the pharmaceutical composition, the injectable solution or the kit as defined above is administered within 48 hours from the onset of brain inflammation and/or after a brain injury, preferably within 24 hours, more preferably within 12 hours.

In some embodiments, the pharmaceutical composition, the injectable solution or the kit as defined above is administered within the first 10 hours from the onset of brain inflammation and/or after a brain injury, preferably within the first 9 hours, more preferably within the first 8 hours, even more preferably within the first 7 hours, even more preferably within the first 6 hours. In other words, in one embodiment, the pharmaceutical composition, the injectable solution or the kit as defined above is administered from about 0 hours to about 120 hours after the onset of brain inflammation and/or after a brain injury, preferably within 96 hours, more preferably within 72 hours, more preferably within 48 hours, more preferably within 24 hours, more preferably within 12 hours, more preferably within 10 hours, more preferably within 9 hours, more preferably within 8 hours, more preferably within 7 hours, more preferably within 6 hours.

Therefore, in some embodiments, the pharmaceutical composition, the injectable solution or the kit as defined above is administered as soon as possible after the onset of brain hemorrhage. In some embodiments, the pharmaceutical composition, the injectable solution or the kit as defined above is administrated to the subject rapidly after a brain hemorrhage. In some embodiments, the pharmaceutical composition, the injectable solution or the kit as defined above is administered within 120 hours after the onset of brain hemorrhage, preferably within 96 hours, more preferably within 72 hours. In some embodiments, the pharmaceutical composition, the injectable solution or the kit as defined above is administered within 48 hours after the onset of brain hemorrhage, preferably within 24 hours, more preferably within 12 hours. In some embodiments, the pharmaceutical composition, the injectable solution or the kit as defined above is administered within the first 10 hours after the onset of brain hemorrhage, preferably within the first 9 hours, more preferably within the first 8 hours, even more preferably within the first 7 hours, even more preferably within the first 6 hours. In other words, in one embodiment, the pharmaceutical composition, the injectable solution or the kit as defined above is administered within about 0 hours to about 120 hours after the onset of brain hemorrhage, preferably within 96 hours, more preferably within 72 hours, more preferably within 48 hours, more preferably within 24 hours, more preferably within 12 hours, more preferably within 10 hours, more preferably within 9 hours, more preferably within 8 hours, more preferably within 7 hours, more preferably within 6 hours.

It is to be understood that the sooner the pharmaceutical composition, the injectable solution or the kit as defined above is administered, the better the clinical outcomes.

Therefore, in some embodiments, the pharmaceutical composition, the injectable solution or the kit as defined above is administered within about 0 hours, 1 hour, 2 hours, 3 hours, 4 hours, 5 hours, 6 hours, 7 hours, 8 hours, 9 hours, 10 hours, 12 hours, 24 hours or 48 hours to about 120 hours, 96 hours or 72 hours after the onset of brain inflammation and/or after a brain injury.

Therefore, in some embodiments, the pharmaceutical composition, the injectable solution or the kit as defined above is administered within about 0 hours, 1 hour, 2 hours, 3 hours, 4 hours, 5 hours, 6 hours, 7 hours, 8 hours, 9 hours, 10 hours, 12 hours, 24 hours or 48 hours to about 120 hours, 96 hours or 72 hours after the onset of brain hemorrhage.

In one embodiment, the pharmaceutical composition, the injectable solution or the kit as defined above is administered from about 24 hours to about 120 hours after the onset of brain inflammation and/or after a brain injury, preferably from about 24 hours to about 96 hours, more preferably from about 24 hours to about 72 hours. In one embodiment, the pharmaceutical composition, the injectable solution or the kit as defined above is administered from about 48 hours to about 120 hours after the onset of brain inflammation and/or after a brain injury, preferably from about 48 hours to about 96 hours, more preferably from about 48 hours to about 72 hours.

In one embodiment, the pharmaceutical composition, the injectable solution or the kit as defined above is administered within about 24 hours to about 120 hours after the onset of brain hemorrhage, preferably within about 24 hours to about 96 hours, more preferably within about 24 hours to about 72 hours. In one embodiment, the pharmaceutical composition, the injectable solution or the kit as defined above is administered within about 48 hours to about 120 hours after the onset of brain hemorrhage, preferably within about 48 hours to about 96 hours, more preferably within about 48 hours to about 72 hours.

In some embodiments, the pharmaceutical composition, the injectable solution or the kit as defined above is administered from about 0 hour to about 72 hours after the onset of brain inflammation and/or after a brain injury, preferably from about 2 hours to about 48 hours, preferably from about 4 hours to about 36 hours, more preferably from about 6 hours to about 12 hours. In some embodiments, the pharmaceutical composition, the injectable solution or the kit as defined above is administered from about 4 hours to about 48 hours after the onset of brain inflammation and/or after a brain injury, preferably from about 4 hours to about 36 hours, more preferably from about 4 hours to about 12 hours, even more preferably from about 4 hours to about 8 hours. In some embodiments, the pharmaceutical composition, the injectable solution or the kit as defined above is administered from about 6 hours to about 48 hours after the onset of brain inflammation and/or after a brain injury, preferably from about 6 hours to about 36 hours, more preferably from about 6 hours to about 12 hours, even more preferably from about 6 hours to about 8 hours.

In some embodiments, the pharmaceutical composition, the injectable solution or the kit as defined above is administered within about 0 hour to about 72 hours after the onset of brain hemorrhage, preferably within about 2 hours to about 48 hours, preferably from about 4 hours to about 36 hours, more preferably within about 6 hours to about 12 hours. In some embodiments, the pharmaceutical composition, the injectable solution or the kit as defined above is administered within about 4 hours to about 48 hours after the onset of brain hemorrhage, preferably within about 4 hours to about 36 hours, more preferably within about 4 hours to about 12 hours, even more preferably within about 4 hours to about 8 hours. In some embodiments, the pharmaceutical composition, the injectable solution or the kit as defined above is administered within about 6 hours to about 48 hours after the onset of brain hemorrhage, preferably within about 6 hours to about 36 hours, more preferably within about 6 hours to about 12 hours, even more preferably within about 6 hours to about 8 hours.

In some embodiments, the pharmaceutical composition, the injectable solution or the kit as defined above is administered from about 0 hours to about 12 hours after the onset of brain inflammation and/or after a brain injury, preferably from about 0 hours to about 10 hours, more preferably from about 0 hours to about 8 hours, even more preferably from about 0 hours to about 7 hours, even more preferably from about 0 hours to about 6 hours.

In some embodiments, the pharmaceutical composition, the injectable solution or the kit as defined above is administered within about 0 hours to about 12 hours after the onset of brain hemorrhage, preferably within about 0 hours to about 10 hours, more preferably within about 0 hours to about 8 hours, even more preferably within about 0 hours to about 7 hours, even more preferably within about 0 hours to about 6 hours.

In some embodiments, the pharmaceutical composition, the injectable solution or the kit as defined above is administered at most 120 hours after the onset of brain inflammation and/or after a brain injury, preferably at most 96 hours, more preferably at most 72 hours. In some embodiments, the pharmaceutical composition, the injectable solution or the kit as defined above is administered at most 48 hours after the onset of brain inflammation and/or after a brain injury, preferably at most 24 hours, more preferably at most 12 hours, even more preferably at most 8 hours, even more preferably at most 7 hours, even more preferably at most 6 hours or less.

In some embodiments, the pharmaceutical composition, the injectable solution or the kit as defined above is administered at most 120 hours after the onset of brain hemorrhage, preferably at most 96 hours, more preferably at most 72 hours. In some embodiments, the pharmaceutical composition, the injectable solution or the kit as defined above is administered at most 48 hours after the onset of brain hemorrhage, preferably at most 24 hours, more preferably at most 12 hours, even more preferably at most 8 hours, even more preferably at most 7 hours, even more preferably at most 6 hours or less.

Thus, in some embodiments, the disease is brain inflammation occurring after a brain injury and/or a brain hemorrhage, preferably an intracerebral hemorrhage, wherein the pharmaceutical composition, injectable solution, or kit as described herein is administered within 120 hours, preferably within 96 hours, more preferably within 72 hours after the onset of the brain injury and/or brain hemorrhage, preferably intracerebral hemorrhage. In certain embodiments, the disease is brain inflammation occurring after an intracerebral hemorrhage, wherein the pharmaceutical composition, injectable solution, or kit as described herein is administered within 120 hours, preferably within 96 hours, more preferably within 72 hours after the onset of the intracerebral hemorrhage.

In some other embodiments, the disease is neuronal cell death occurring after a brain injury and/or a brain hemorrhage, preferably an intracerebral hemorrhage, wherein the pharmaceutical composition, injectable solution, or kit as described herein is administered within 120 hours, preferably within 96 hours, more preferably within 72 hours after the onset of the brain injury and/or brain hemorrhage, preferably intracerebral hemorrhage. In certain embodiments, the disease is neuronal cell death occurring after an intracerebral hemorrhage, wherein the pharmaceutical composition, injectable solution, or kit as described herein is administered within 120 hours, preferably within 96 hours, more preferably within 72 hours after the onset of the intracerebral hemorrhage.

In some embodiments, the pharmaceutical composition, injectable solution, or the kit as described herein, is for use for inhibiting the intrinsic coagulation pathway in a subject in need thereof as described hereinabove.

In some embodiments, the pharmaceutical composition, injectable solution, or the kit as described herein, is for use for inhibiting platelet recruitment, neutrophil recruitment, neutrophil activation and/or NETosis.

It will be understood that the total daily usage of the protein of the pharmaceutical composition, the injectable solution or the kit as defined above will be decided by the attending physician within the scope of sound medical judgment. The specific therapeutically effective dose level for any particular patient will depend upon a variety of factors including the disorder being treated and the severity of the disorder; activity of the specific agent employed; the specific composition employed, the age, body weight, general health, sex and diet of the patient; the time of administration, route of administration, and rate of excretion of the specific agent employed; the duration of the treatment; drugs used in combination or coincidental with the specific agent employed; and like factors well known in the medical arts. For example, it is well within the skill of the art to start doses of the agent at levels lower than those required to achieve the desired therapeutic effect and to gradually increase the dosage until the desired effect is achieved. However, the daily dosage of the products may be varied over a wide range from about 10 to about 10000 mg per adult per day, preferably 100 to about 5000, more preferably from about 200 to about 2000 mg per adult per day. Preferably, the compositions contain 10, 50, 100, 250, 500, 1000 and 2,000 mg of the active ingredient for the symptomatic adjustment of the dosage to the patient to be treated. A medicament typically contains from about 10 to about 10000 mg of the active ingredient, preferably 5 to about 5000, more preferably from about 10 to about 2000 mg of the active ingredient. An effective amount of the drug is ordinarily supplied at a dosage level from 0.01 mg/kg to about 100 mg/kg of body weight per day, preferably from about 0.05 mg/kg to 40 mg/kg of body weight per day, more preferably from about 0.1 mg/kg to 20 mg/kg of body weight per day, more preferably from about 0.2 mg/kg to 1 mg/kg of body weight per day.

In one embodiment, pharmaceutical composition, kit, injectable composition or medicament of the invention is in a form adapted for injection, such as, for example, for intraocular, intramuscular, subcutaneous, intradermal, transdermal or intravenous injection or infusion.

Examples of forms adapted for injection include, but are not limited to, solutions, such as, for example, sterile aqueous solutions, or forms suitable for using to prepare solutions upon the addition of a liquid prior to use, such as, for example physiological liquid.

In a particular embodiment, the pharmaceutical composition, kit, injectable solution or medicament of the invention is in a form adapted for intraocular administration.

In one embodiment, the pharmaceutical composition, kit, injectable solution or medicament of the invention is administered to the subject in need thereof at least once a day. For example, the pharmaceutical composition, kit, injectable solution or medicament of the invention may be administered once a day, twice a day, or three times a day. In a preferred embodiment, the pharmaceutical composition, kit, injectable solution or medicament of the invention is administered to the subject in need thereof once a day.

In another embodiment, the pharmaceutical composition, kit, injectable solution or medicament of the invention is administered to the subject in need thereof at least once a week. For example, the composition, pharmaceutical composition, or medicament of the invention may be administered once a week, twice a week, three times a week, four times a week or up to seven times a week.

In another embodiment, the pharmaceutical composition, kit, injectable solution or medicament of the invention is administered to the subject in need thereof once a month, two times a month, every two months, every two or three months, two times a year or once a year.

In one embodiment, the pharmaceutical composition, kit, injectable solution or medicament of the invention is administered to the subject in need thereof before being exposed to a risk to develop thrombus. In one embodiment, the term “before” means at least a week before the exposure. In another embodiment, the term “before” means five days, four days, three days, two days or one day before the exposure. In another embodiment, the term “before” means 24 hours, 18 hours, 15 hours, 12 hours, 6 hours, 4 hours, 2 hours or 1 hour before the exposure. In another embodiment, the term “before” means less than one hour before the exposure, such as 45 minutes, 30 minutes, 15 minutes, 10 minutes, 5 minutes before the exposure or at the moment of the exposure. As an illustration, the composition, pharmaceutical composition, or medicament of the invention may be administered to the subject 24 hours, 12 hours, 6 hours or 1 hour before a prolonged travel, or at the beginning of the prolonged travel.

In another embodiment, the pharmaceutical composition, kit, injectable solution or medicament of the invention is administered to the subject in need thereof after being exposed to a risk to develop thrombus. In one embodiment, the term “after” means 5 minutes, 10 minutes, 15 minutes, 30 minutes, or 45 minutes after the exposure. In another embodiment, the term “after” means 1 hour, 2, 4, 6, 12, 15, 18 or 14 hours after the exposure. In another embodiment, the term “after” means 1 day, 2, 3, 4 or five days after the exposure. In another embodiment, the term “after” means a week or more after the exposure. As an illustration, the composition, pharmaceutical composition, or medicament of the invention may be administered to the subject immediately after a prolonged travel, 1 hour, 2 hours, 6 hours, or 12 hours after the prolonged travel.

The invention further relates to the use of the pharmaceutical composition, the injectable solution or the kit as defined above, for the manufacture of a medicament. The invention further relates to the use of the pharmaceutical composition, the injectable solution or the kit as defined above, for the manufacture of a medicament for treating a disease as described herein.

The invention further relates to a method of treatment comprising administering to a subject the pharmaceutical composition or the injectable solution as defined above. The invention further relates to a method of treating and/or reducing the likelihood of occurrence of a disease as described herein, comprising administering to a subject a therapeutically effective dose of the pharmaceutical composition or the injectable solution as defined above.

In one embodiment, the pharmaceutical composition, the injectable solution, the kit, or the medicament of the invention comprises a therapeutically effective amount of the polypeptide that has at least 75% sequence identity with the amino acid sequence SEQ ID NO: 2.

It will be understood that the total daily usage of the pharmaceutical composition, injectable solution, or the kit according to the invention will be decided by the attending physician within the scope of sound medical judgment. The specific therapeutically effective dose level for any particular patient will depend upon a variety of factors including the disorder being treated and the severity of the brain inflammation; activity of the specific agent employed; the specific composition employed, the age, body weight, general health, sex and diet of the patient; the time of administration, route of administration, and rate of excretion of the specific agent employed; the duration of the treatment; drugs used in combination or coincidental with the specific agent employed; and like factors well known in the medical arts. For example, it is well within the skill of the art to start doses of the agent at levels lower than those required to achieve the desired therapeutic effect and to gradually increase the dosage until the desired effect is achieved.

However, the daily dosage of the products may be varied over a wide range from about 10 to about 10000 mg per adult per day, preferably 100 to about 5000, more preferably from about 200 to about 2000 mg per adult per day. Preferably, the compositions contain 10, 50, 100, 250, 500, 1000 and 2,000 mg of the active ingredient (i.e., the protein or polypeptide having at least 75% identity with SEQ ID NO: 2, also referred to as Ir—CPI or BIOX-101) for the symptomatic adjustment of the dosage to the patient to be treated. A medicament typically contains from about 10 to about 10000 mg of the active ingredient, preferably 5 to about 5000, more preferably from about 10 to about 2000 mg of the active ingredient. An effective amount of the drug is ordinarily supplied at a dosage level from 0.01 mg/kg to about 100 mg/kg of body weight per day, preferably from about 0.05 mg/kg to 40 mg/kg of body weight per day, more preferably from about 0.1 mg/kg to 20 mg/kg of body weight per day, more preferably from about 0.2 mg/kg to 1 mg/kg of body weight per day.

In some embodiments, the pharmaceutical composition, injectable solution, or the kit according to the invention is administered at a dose from about 2.5 mg/kg/day to about 250 mg/kg/day, preferably from about 2.5 mg/kg/day to about 100 mg/kg/day, more preferably from about 2.5 mg/kg/day to about 50 mg/kg/day, more preferably from about 2.5 mg/kg/day to about 25 mg/kg/day. In some embodiments, the pharmaceutical composition, injectable solution, or the kit according to the invention is administered at a dose from about 5 mg/kg/day to about 50 mg/kg/day. In some embodiments, the pharmaceutical composition, injectable solution, or the kit according to the invention is administered at a dose from about 10 mg/kg/day to about 50 mg/kg/day. In some embodiments, the pharmaceutical composition, injectable solution, or the kit according to the invention is administered at a dose of about 25 mg/kg/day.

In some embodiments, the pharmaceutical composition, injectable solution, or the kit according to the invention is administered at a dose from about 20 mg to about 200,000 mg per adult per day, preferably from about 20 mg to about 20,000 mg per adult per day, more preferably from about 20 mg to about 10,000 mg per adult per day. In some embodiments, the pharmaceutical composition, injectable solution, or the kit according to the invention is administered at a dose from about 200 mg to about 200,000 mg per adult per day, preferably from about 200 mg to about 20,000 mg per adult per day, more preferably from about 200 mg to about 10,000 mg per adult per day. In some embodiments, the pharmaceutical composition, injectable solution, or the kit according to the invention is administered at a dose of about 2000 mg per adult per day.

In a preferred embodiment, the protein or polypeptide comprising a polypeptide that has at least 75% sequence identity with the amino acid sequence SEQ ID NO: 2 is present in the pharmaceutical composition as described herein at a concentration from 15 mg/mL to 35 mg/mL, preferably from 20 mg/mL to 30 mg/mL, preferably around 25 mg/mL.

In a preferred embodiment, the protein or polypeptide comprising a polypeptide that has at least 75% sequence identity with the amino acid sequence SEQ ID NO: 2 is present in the injectable solution as described herein at a concentration from 2 mg/mL to 10 mg/mL, preferably from 3 mg/mL to 7.5 mg/mL, preferably from 4 mg/mL to 6 mg/mL, preferably around 5 mg/mL.

In some embodiments, the concentration of the stabilizing agent is from 1 mM to 50 mM, preferably from 10 mM to 40 mM, preferably from 15 mM to 30 mM.

In one embodiment, the pharmaceutical composition, injectable solution, or the kit according to the invention is administered to the subject in need thereof at least once a day. For example, the composition, pharmaceutical composition, or medicament of the invention may be administered once a day, twice a day, or three times a day. In a preferred embodiment, the composition, pharmaceutical composition, or medicament of the invention is administered to the subject in need thereof once a day.

In another embodiment, the pharmaceutical composition, injectable solution, or the kit according to the invention is administered to the subject in need thereof at least once a week. For example, the composition, pharmaceutical composition, or medicament of the invention may be administered once a week, twice a week, three times a week, four times a week or up to seven times a week.

In another embodiment, the pharmaceutical composition, injectable solution, or the kit according to the invention is administered to the subject in need thereof once a month, two times a month, every two months, every two or three months, two times a year or once a year.

In one embodiment, the pharmaceutical composition, injectable solution, or the kit according to the invention is administered to the subject in need thereof before being exposed to a risk to develop brain inflammation. In one embodiment, the term “before” means at least a week before the exposure. In another embodiment, the term “before” means five days, four days, three days, two days or one day before the exposure. In another embodiment, the term “before” means 24 hours, 18 hours, 15 hours, 12 hours, 6 hours, 4 hours, 2 hours or 1 hour before the exposure. In another embodiment, the term “before” means less than one hour before the exposure, such as 45 minutes, 30 minutes, 15 minutes, 10 minutes, 5 minutes before the exposure or at the moment of the exposure.

In another embodiment, the pharmaceutical composition, injectable solution, or the kit according to the invention is administered to the subject in need thereof after being exposed to a risk to develop brain inflammation. In one embodiment, the term “after” means 5 minutes, 10 minutes, 15 minutes, 30 minutes, or 45 minutes after the exposure.

In another embodiment, the term “after” means 1 hour, 2, 4, 6, 12, 15, 18 or 14 hours after the exposure. In another embodiment, the term “after” means 1 day, 2, 3, 4 or five days after the exposure. In another embodiment, the term “after” means a week or more after the exposure.

In some embodiments, the pharmaceutical composition, injectable solution, or the kit according to the invention is administered continuously, typically it is administered by perfusion.

In some embodiments, the pharmaceutical composition, the injectable solution or the kit as defined above is administered continuously, preferably by perfusion, for about 12 hours to about 96 hours, preferably for about 18 hours to about 84 hours, more preferably for about 12 hours to about 72 hours. In some embodiments, the pharmaceutical composition, the injectable solution or the kit as defined above is administered continuously, preferably by perfusion, for about 24 hours to about 96 hours, preferably for about 48 hours to about 96 hours. In some embodiments, the pharmaceutical composition, the injectable solution or the kit as defined above is administered continuously, preferably by perfusion, for about 24 hours to about 72 hours, preferably for about 48 hours to about 72 hours.

In some embodiments, the pharmaceutical composition, the injectable solution or the kit as defined above is administered continuously, preferably by perfusion, for at least about 12 hours, 24 hours, 36 hours, 48 hours, 72 hours or 96 hours. In a particular embodiment, the pharmaceutical composition, the injectable solution or the kit as defined above is administered continuously, preferably by perfusion, for at least 48 hours.

In some embodiments, the subject is administered with a first dose of the pharmaceutical composition, injectable solution, or the kit according to the invention, and is then perfused with the same for continuous administration. Within the scope of the present invention, the first dose is hereby referred to as the initial dose. In some embodiments, the dosage of the pharmaceutical composition, injectable solution, or the kit according to the invention in the initial dose is higher or equal, preferably higher, than the dosage in the perfusion.

### EXAMPLES

The present invention is further illustrated by the following examples.

### Example 1-Formulation 1

### Manufacturing Process of Recombinant Ir—CPI in Pichia (Komagataella) Pastoris

Host strain for the working cell bank: Pichia (Komagataella) pastoris CBS 7435 mutS. VTU Technology has generated a mutated strain of the wildtype strain Pichia pastoris CBS 7435 to create a basic expression strain with slower methanol use (mutS), called P. pastoris CBS 7435 mutS.
Clone: 32F5.
Plasmids: pPZ-alpha-V0.1/pPK-KEX2.
Upstream Process (Fermentation)

### Step 1: Seed Thawing and Pre-Cultures

Thawing: The working cell bank is thawed at room temperature for 5-15 minutes. Pre-Culture I: Inoculate shake flasks with YPhyG medium containing the working cell bank; incubate at 28 °C, 180 rpm until OD600≥8. Check for contamination via microscopy. Pre-Culture II: Transfer to a bioreactor; batch fermentation at pH 5.05, 28 °C, with DO>5%. Once OD600≥8 is reached and confirmed clean by microscopy, proceed to main culture.

### Step 2: Fermentation

Batch Phase: Inoculate a bioreactor (containing BSM medium) with pre-culture II. Maintain: pH 5.05±0.3, temperature 28 °C, DO>5%.

Glycerol Fed-Batch Phase: Glycerol used as carbon source. A pO2 spike signals glycerol depletion. Begin exponential glycerol feed (μ=0.01 h⁻¹, starting at 3.18 kg/h); after 1 hour, maintain a constant rate of 25.4 kg for 4 hours.

Methanol Fed-Batch Phase: After 3 hours of glycerol feeding: gradually reduce temperature to 24 °C, increase pH to 5.55. Once conditions are adjusted and glycerol is consumed, switch to methanol feed. Start methanol feeding after second pO2 spike; gradually increase from 1.000 to 3.429 kg/h over 66 h, then hold at 3.429 kg/h. Harvest: 90 h after starting methanol feed. Confirm absence of contamination. Upon need, antifoam is added.

### Step 3: Cooling

After methanol feeding: cool bioreactor to 15±1 °C. Stop feeding and pH/DO regulation. Set stirrer to 200 rpm, aeration to 550 L/min, and apply 1 bar backpressure.

### Step 4: Harvest and Clarification

Biomass removal: Supernatant containing the target protein is separated from cells by centrifugation. Further clarification: The supernatant is refined by a second centrifugation (15 °C).

### Step 5: pH and Conductivity Adjustment

pH adjustment: Set to 5.50±0.10 using glacial acetic acid (HAc). Conductivity adjustment: Diluted with RO—I water to achieve 5.0±0.5 mS/cm.

### Step 6: Filtration

Depth filtration: Performed using a Millistak+ HC Pod, pre-rinsed with RO—I water. Final filtration: Tangential Flow Filtration (TFF) through a 0.2 μm hollow fiber membrane. Post-use rinse: Membrane is cleaned with water for injection (WFI).

### Downstream Process

Step 7: Fractogel EMD DEAE chromatography

Anion exchange chromatography (AEC) is conducted with Fractogel EMD DEAE resin packed into a BPG 300 column. The dimensions of the packed bed are: diameter=35 cm, cross-sectional area=962 cm², height=21.5 cm (20.5-22.5 cm), packed volume=14.8 L (14.1-15.5 L). The chromatography is performed using a 10 mm Millipore K-Prime 40-II system.

### Step 8: SP Sepharose FF Chromatography

Anion exchange chromatography is conducted with SP Sepharose FF gel packed in a Quikscale 450 column. The SP Sepharose FF step is performed using a 10 mm Millipore K-Prime 40-III system. After the first SP Sepharose step, the eluate fraction (SPFF1-E2 fraction) is stored at +2/+8 °C for ≤72 h.

### Step 9: Ultrafiltration

Cycle 1—Ultrafiltration of the Eluate. Equipment: 6 Sartorius Sartocon PESU membranes (5 kDa), mounted in a Sartorius holder and connected to a Pall Centrasette system. Process: Concentrate the eluate 5-fold. Diafilter with 5 volumes of PBS pH 7.4. Flush product out of the system. Perform two rinses with the same buffer to recover the remaining product.

### Step 10: Formulation 1

The concentration is adjusted to 25.0±2.5 mg/mL. Based on the total concentration in individual washouts, it is calculated how much washout must be added to the retentate. The washout is added to the retentate while stirring slightly. If the final concentration in the final retentate has not been reached, it is adjusted with a calculated amount of PBS pH 7.4. The targeted concentration is measured in UV at 280 nm.

### Example 2—Alternative Formulations with Additives

A number of new formulations were prepared starting from Formulation 1 by addition of methionine or L-Arginine as follows:

Formulation 2:150 μL of concentrated methionine solution was added to 5 mL Formulation 1 to reach a final protein concentration of 24.3 mg/mL, a final methionine concentration of 10 mM and pH 7.4:

Formulation 3:150 μL of concentrated L-arginine solution was dissolved to 5 mL Formulation 1 to reach a final protein concentration of 24.3 mg/mL, a final arginine concentration of 50 mM and pH 7.4.

### Example 3—Formulation 4

Manufacturing process of Ir—CPI Drug Substance (SEQ ID NO:2) in histidine buffer (production in P. pastoris).

Formulation 1 was buffer exchanged using Vivaspin® 6 centrifugal filter (from PBS to L-histidine). Four centrifugation cycles were performed (4000 g×20 min each). The pH of the solution was adjusted to 6.0 by addition of HCl (37%), yielding Formulation 4 having a final protein concentration of 23.2 mg/mL, a final L-Histidine concentration of 20 mM and pH 6.0.

### Example 3B

Alternatively, the formulation of Example 3 can be obtained with less steps, by using directly an aqueous solution of L-Histidine at a concentration of 20 mM and pH 6.0 in steps 9 and 10 of Example 1.

### Example 4—Evaluation of Buffer/Additive Effect on Ir—CPI Stability: Temperature Ramp Study

### Materials and Method

The purpose is to assess the conformational stability of Ir—CPI formulation candidates using a temperature ramp (thermal stress).

The method of dynamic light scattering is the most common measurement technique for particle size analysis in the nanometer range. Dynamic light scattering is based on the Brownian motion of dispersed particles. When particles are dispersed in a liquid they move randomly in all directions. The principle of Brownian motion is that particles are constantly colliding with solvent molecules. These collisions cause a certain amount of energy to be transferred, which induces particle movement. Energy transfer is more or less constant and therefore has a greater effect on smaller particles. As a result, smaller particles are moving at higher speeds than larger particles. Knowing the other parameters which have an influence on particle movement, it is possible to determine the hydrodynamic diameter by measuring the speed of the particles. The relation between the speed of the particles and the particle size is given by the Stokes-Einstein equation.

Concretely, a single frequency laser is directed to the sample contained in a cuvette. If there are particles in the sample, the incident laser light gets scattered in all directions. The scattered light is detected at a certain angle over time and this signal is used to determine the diffusion coefficient and the particle size by the Stokes-Einstein equation.

The first order result from a DLS experiment is the intensity distribution of particle sizes. The intensity distribution is weighted according to the scattering intensity of each particle fraction or family. The particle scattering intensity is proportional to the square of the molecular weight. The intensity distribution gives us the amount of light scattered by particles in the diameter range of x to x+Δx. As such, the intensity distribution can be somewhat misleading, in that a small amount of aggregates or larger particles can dominate the distribution. In typical applications, a result that is more desirable than the intensity distribution is either the volume (equivalent to mass/weight distribution) or number distribution to estimate the relative amount for each separate peak.

Note that in Size Distribution by Intensity, large particles generate a greater response as the intensity is proportional to the particle diameter to the ninth. In comparison, number-based distribution is directly proportional to the particle diameter and volume-based distributions are proportional to the particle diameter to the third.

### Results

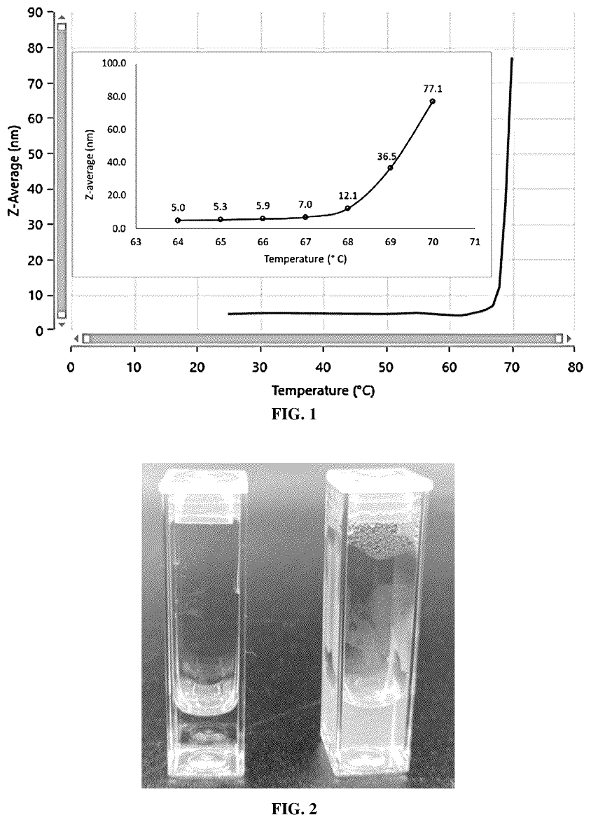

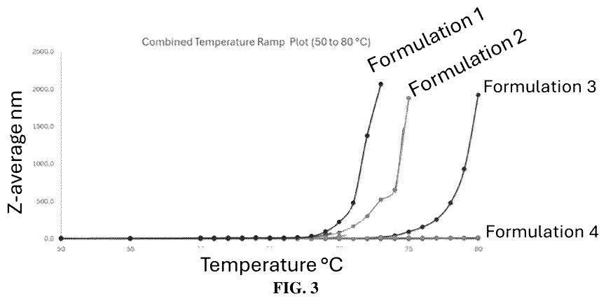

The onset temperature for aggregation has been observed at around 67 °C for Formulation 1 (Ir—CPI batch B3015310) formulated at 25 mg/mL in PBS. The appearance of the sample changed from a clear solution to an opalescent one at the end of the temperature ramp experiment (FIG. 2). The Z-average increased from 5.3 nm at ~66 °C to 77.1 nm at ~70 °C (FIG. 1).

Formulation 1 was then compared with Formulations 2 to 4. As shown in FIG. 3, Formulation 3 (L-arginine) and Formulation 2 (L-methionine) delayed but did not prevent aggregation in the temperature ramp study while no aggregation was observed with Formulation 4 (L-histidine) in the tested conditions. Histidine also prevented opalescence appearance in the temperature ramp study.

### Example 5—Stability Studies

### Materials and Method

Stability of Formulations 1 (PBS) and 4 (L-Histidine) was evaluated at 25 °C/60% RH (relative humidity) and at 30 °C/65% RH as well as a freeze/thaw study.

The vials containing the Formulations were retrieved from the −20 °C freezer and thawed at room temperature for ~3 hours. Each vial was opened and pooled (5 mL serological pipettes). For Formulation 1 (control), 10 mL of the pool were filtered (Low Protein binding Durapore PVDF membrane, 0.2 μm, 33 mm) and filled into 2 vials (10 mL vials filled with 5 ml). Formulation 4 was prepared from Formulation 1, buffer exchanges were done using Slide-A-Lyzer (G3 dialysis cassette, 3.5 kDa, 125 mL). Membranes were hydrated for 2 min and approximately 90-92 mL of Ir—CPI was added. The buffer (1.0 L) was replaced three times i.e., after 2 hours, 5 hours and after dialyzing overnight. Formulation 4 was recovered from the dialysis cassette, filtered and filled into vials for TO analysis.

Time points tested are presented in Table 1:

TABLE 1
Stability time points tested on the 2 formulations
stored at 25 °C/60% RH or 30 °C/65% RH.

| Storage | Storage time |
|---|---|
| conditions | (weeks) |
| 25 °C/60% RH | T0 |
| | T8 |
| | T11 |
| | T12 |
| | T16 |
| 30 °C/65% RH | T0 |
| | T5 |
| | T6 |
| | T7 |
| | T8 |
| | T13 |
| | T14 |

When a formulation became opalescent, further timepoints were not tested anymore.

### Results

All formulations met specifications at TO (Table 2).

TABLE 2
Specification and Analytical Comparison of Ir-
CPI in PBS and Histidine Formulations at T0.

| Parameter | Specification | PBS | Histidine |
|---|---|---|---|
| Appearance: | Clear to slightly | Clear | Clear |
| clarity and | opalescent (not | transparent | transparent |
| degree of | more turbid than | liquid | liquid |
| opalescence | the reference | | |
| | suspension III) | | |
| Visible particles | Practically free of | Few dust | Few dust |
| | visible particles | particles | particles |
| Sub visible | <6000/container | 377 | 63 |
| particles ≥10 μm | | | |
| Sub visible | <600/container | 13 | 3 |
| particles ≥25 μm | | | |
| DLS particle size | Report result | 5.38 | 3.312 |
| (nm) | | | |
| Activity (aPTT | Comparable to reference | 1.88 | 1.89 |
| ratio at 0.25 μM) | (1.93) ± 20% | | |
| UV (mg/mL) | 25 mg/mL ± 10% | 24.1 | 22.2 |
| UV (A350/A280) | <0.1 | <0.1 | <0.1 |
| Purity by RP- | Main peak (at 3.9 | 100% Purity | 100% Purity |
| HPLC | min) >95% | Single peak | Single peak |
| | | at 3.98 min | at 3.98 min |
| Impurity by SE- | Ratio dimer/ | Single peak | Single peak |
| HPLC | monomer <15% | at 36.2 min | at 36.2 min |
| Purity by SDS- | 1 band | Single band | Single band |
| PAGE | | | |

Results of analysis after 3 Freeze/Thaw cycles (F/T cycles) are presented in Table 3. Note that the average particle size was slightly higher in Formulation 1 than in Formulation 4 at TO and after the 3 F/T cycles.

TABLE 3
Comparative stability profile of Formulation
1 and 4 at T0 and after 3 Freeze/Thaw cycles.

| | Formulation 1 | | Formulation 4 | |
|---|---|---|---|---|
| Parameters | T0 | 3 × F/T | T0 | 3 × F/T |
| Appearance: | Clear | Clear | Clear | Clear |
| clarity and | transparent | transparent | transparent | transparent |
| degree of | liquid | liquid | liquid | liquid |
| opalescence | | | | |
| Visible | Few dust | Few dust | Few dust | No visible |
| particles | particles | particles | particles | particles |
| Sub visible | 377 | 130 | 63 | 210 |
| particles ≥10 | | | | |
| μm | | | | |
| Sub visible | 13 | 0 | 3 | 67 |
| particles ≥25 | | | | |
| μm | | | | |
| DLS particle | 5.38 | 5.483 | 3.312 | 3.351 |
| size (nm) | | | | |
| Activity (aPTT | 1.88 | 1.90 | 1.89 | 1.90 |
| ratio at | | | | |
| 0.25 μM) | | | | |

Both formulations remained clear and transparent after three freeze-thaw (F/T) cycles. No product-related visible particles were observed at either TO or after 3 F/T cycles. The number of subvisible particles fell within Ph.Eur. requirements for both formulations at TO and after three freeze thaw cycles. Activity as measured by aPTT remained stable. No significant difference in DLS average particle size for both formulations was observed between TO and after the 3 F/T cycles.

### Stability of the Formulations at 25 °C and 30 °C.

For Formulation 1 at 25 °C/60% RH, opalescence appeared between T8 and T11 weeks (Table 4). At 30 °C/65% RH, opalescence and aggregation were visible at T5 weeks, with a decrease of activated Partial Thromboplastin Time (aPTT) and an increase of DLS particle size (Table 5).

TABLE 4
Stability data on Formulation 1 (Ir-CPI formulated
in PBS) over 11 weeks at 25 °C/60% RH.

| 25 °C/60% RH | T0 | T8 | T11 |
|---|---|---|---|
| Appearance: | Clear | Clear | White turbid liquid |
| clarity and | transparent | transparent | with caking white |
| opalescence | liquid | liquid | sediment |

TABLE 5
Stability data on Formulation 1 (Ir-CPI formulated
in PBS) over 5 weeks at 30 °C/65% RH.

| 30 °C/65% RH | T0 | T5 |
|---|---|---|
| Appearance | Clear transparent | White turbid liquid with |
| | liquid | caking white sediment |
| Visible particles | No visible particles | |
| Sub visible | 377 | 300 |
| particles ≥10 μm | | |
| Sub visible | 13 | 27 |
| particles ≥25 μm | | |
| DLS particle size (nm) | 5.38 | 310 |
| Activity (aPTT ratio | 1.88 (1.93) | 1.78 (2.04) |
| at 0.25 μM) | | |

Regarding Formulation 4, at 25 °C/60% RH, the solution remained stable till 16 weeks (end of the program) when considering appearance, visible, sub-visible and DLS particles as well as aPTT (Table 6). After 5 weeks of storage at 30 °C/65% RH, Formulation 4 remained stable until T13 weeks (end of stability program, Table 7).

TABLE 6
Stability data of Formulation 4 ([Ir-CPI formulated in histidine
buffer (20 mM, pH 6.0)] stored over 16 weeks at 25 °C/60% RH.
Histidine 20 mM pH 6.0

| 25 °C/60% RH | T0 | T8 | T11 | T12 | T16 |
|---|---|---|---|---|---|
| Appearance: clarity | Clear | Clear | Clear | Clear | Clear |
| and degree of | transparent | transparent | transparent | transparent | transparent |
| opalescence | liquid | liquid | liquid | liquid | liquid |
| Visible particles | Few dust | | | | No visible |
| | particles | | | | particles |
| Sub visible | 63 | | | | 137 |
| particles ≥10 μm | | | | | |
| Sub visible | 3 | | | | 10 |
| particles ≥25 μm | | | | | |
| DLS particle | 3.312 | | | 20.75 | 3.88 |
| size (nm) | | | | | |
| Activity (aPTT | 1.89 (1.93) | | | 1.93 (2.04) | 1.91 |
| ratio at 0.25 μM) | | | | | |

TABLE 7
Stability data of Formulation 4 (Ir-CPI formulated in histidine buffer (20 mM, pH 6.0)) stored over 13 weeks at 30 °C/65% RH.
Histidine 20 mM pH 6.0

| 30 °C/65% RH | T0 | T5 | T6 | T7 | T8 | T9 | T10 | T11* | T12 | T13 |
|---|---|---|---|---|---|---|---|---|---|---|
| Appearance: | Clear | Clear | Clear | Clear | Clear | Clear | Clear | Clear | Clear | Clear |
| clarity | trans- | trans- | trans- | trans- | trans- | trans- | trans- | trans- | trans- | trans- |
| and | parent | parent | parent | parent | parent | parent | parent | parent | parent | parent |
| opalescence | liquid | liquid | liquid | liquid | liquid | liquid | liquid | liquid | liquid | liquid |
| Visible | Few | No | | | | | | No | | No |
| particles | dust | visible | | | | | | visible | | visible |
| | particles | particles | | | | | | particles | | particles |
| Sub | 63 | 67 | | | | | | 287 | | 60 |
| visible | | | | | | | | | | |
| particles | | | | | | | | | | |
| ≥10 μm | | | | | | | | | | |
| Sub | 3 | 3 | | | | | | 10 | | 10 |
| visible | | | | | | | | | | |
| particles | | | | | | | | | | |
| ≥25 μm | | | | | | | | | | |
| DLS | 3.312 | 3.83 | 4.043 | 4.236 | 4.363 | 4.599 | 4.749 | 6.709 | 5.182 | 5.335 |
| particle size | | | | | | | | | | |
| (nm) | | | | | | | | | | |
| Activity | 1, 89 | 1, 80 | 1, 74 | 1, 65 | 1, 66 | 1, 42 | 1, 36 | 1, 35 | 1, 40 | 1, 25 |
| (aPTT ratio at | (1.93) | (2.04) | (2.04) | (2.04) | (2.04) | (2.04) | (2.04) | (2.04) | (2.04) | (2.04) |
| 0.25 μM) | | | | | | | | | | |
| UV | 22.2 | 23.1 | | | | 24.2 | | | | 23.0 |
| (mg/mL) | | | | | | | | | | |
| UV | <0.1 | <0.1 | | | | <0.1 | | | | <0.1 |
| (A350/A280) | | | | | | | | | | |
| SDS-PAGE | Single | Single | | | | Single | | | | Single |
| (Purity) | band | band | | | | band | | | | band |

1. A pharmaceutical composition comprising:
a protein or polypeptide comprising one polypeptide that has at least 75% sequence identity with the amino acid sequence SEQ ID NO: 2, and
a stabilizing agent of formula (I) to prevent aggregation and/or opalescence 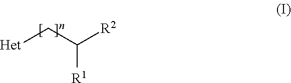
wherein R¹ is H, NH₂ or alanine; R² is H or COOH;
n=0, 1 or 2;
Het is a nitrogen containing heterocycle selected from the group consisting of pyrazol-3-yl, pyrazol-4-yl, pyrazol-5-yl, imidazole-4-yl, 1,2,4-Triazol-3-yl, indol-3-yl, 4-pyridyl, 3-pyridyl, and 2-pyridyl.

2. The pharmaceutical composition according to claim 1, wherein the stabilizing agent is a compound selected from the group consisting of phenylalanine, tyrosine, histidine, histamine, tryptophane, β-(1,2,4-Triazol-3-yl)-alanine, imidazole-4-acetic acid, carnosine, imidazolepropionic acid and 4-pyridyl alanine.

3. The pharmaceutical composition according to claim 1, wherein the stabilizing agent is a compound of formula (II),

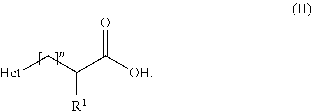

4. The pharmaceutical composition according to claim 1, wherein the stabilizing agent is an amino acid.

5. The pharmaceutical composition according to claim 1, wherein the stabilizing agent is an amino acid in L-configuration.

6. The pharmaceutical composition according to claim 1, wherein the stabilizing agent is histidine.

7. The pharmaceutical composition according to claim 1, wherein the stabilizing agent is L-histidine.

8. The pharmaceutical composition according to claim 1, wherein the concentration of the stabilizing agent is from 1 mM to 50 mM.

9. The pharmaceutical composition according to claim 1, wherein the concentration of the stabilizing agent is from 15 mM to 30 mM.

10. The pharmaceutical composition according to claim 1, having a pH from 5.0 to 7.5.

11. The pharmaceutical composition according to claim 1, having a pH from 5.5 to 7.0.

12. The pharmaceutical composition according to claim 1, wherein the protein or polypeptide comprising a polypeptide that has at least 75% sequence identity with the amino acid sequence SEQ ID NO: 2 is present at a concentration from 15 mg/mL to 35 mg/mL.

13. The pharmaceutical composition according to claim 1, wherein said protein or polypeptide has the amino acid sequence SEQ ID NO: 2; and wherein the stabilizing agent is histidine.

14. An injectable solution comprising:
the pharmaceutical composition according to claim 1, and
a physiological liquid or saline solution.

15. The injectable solution of claim 14, wherein the protein or polypeptide comprising a polypeptide that has at least 75% sequence identity with the amino acid sequence SEQ ID NO: 2 is present at a concentration from 2 mg/mL to 10 mg/mL.

16. The injectable solution of claim 14, wherein the protein or polypeptide comprising a polypeptide that has at least 75% sequence identity with the amino acid sequence SEQ ID NO: 2 is present at a concentration of around 5 mg/mL.

17. A method of treating and/or reducing the likelihood of occurrence of a disease in a subject, comprising administering to said subject a therapeutically effective dose of the pharmaceutical composition according to claim 1, an injectable solution comprising the pharmaceutical composition and a physiological liquid or saline solution, or a kit comprising the pharmaceutical composition comprising the pharmaceutical composition and a physiological liquid or saline solution.

18. The method according to claim 17, wherein said disease is selected from the group consisting of:
thrombotic disorders;
cerebrovascular diseases;
atherosclerosis;
conditions associated with thrombosis;
inflammatory disorders associated with thrombosis;
thromboinflammation;
brain inflammation occurring after a brain injury and/or a brain hemorrhage; and
neuronal cell death occurring after a brain injury and/or a brain hemorrhage.

19. The method according to claim 17, wherein said disease is selected from the group consisting of:
thrombotic disorders selected from the group consisting of venous thrombosis, arterial thrombosis, and thromboembolism;
cerebrovascular diseases selected from the group consisting of ischemic stroke, hemorrhagic stroke, thrombus formation following cerebral injuries, and cerebral ischemia;
atherosclerosis;
conditions associated with thrombosis selected from the group consisting of preeclampsia, eclampsia, and HELLP syndrome; and
inflammatory disorders associated with thrombosis selected from the group consisting of rheumatoid arthritis, sepsis, and inflammatory bowel disease;
thromboinflammation;
brain inflammation occurring after a brain injury and/or a brain hemorrhage, wherein the pharmaceutical composition, the injectable solution, or the kit is administered within 120 hours, 96 hours or 72 hours after the onset of brain injury and/or brain hemorrhage; and
neuronal cell death occurring after a brain injury and/or a brain hemorrhage, wherein the pharmaceutical composition, the injectable solution, or the kit is administered within 120 hours, 96 hours or 72 hours after the onset of brain injury and/or brain hemorrhage.

20. The method according to claim 17, wherein said brain hemorrhage is intracerebral hemorrhage.
